(19) United States
(12) Patent Application Publication
(10) Pub. No.: US 2025/0009329 A1
(43) Pub. Date: Jan. 9, 2025

(54) IMAGE PROCESSING APPARATUS, MEDICAL DIAGNOSTIC APPARATUS, ULTRASONIC ENDOSCOPE APPARATUS, IMAGE PROCESSING METHOD, AND PROGRAM

(71) Applicant: FUJIFILM CORPORATION, Tokyo (JP)

(72) Inventor: Rito MURASE, Kanagawa (JP)

(21) Appl. No.: 18/890,704

(22) Filed: Sep. 19, 2024

Related U.S. Application Data
(63) Continuation of application No. PCT/JP2023/004985, filed on Feb. 14, 2023.

(30) Foreign Application Priority Data
Mar. 29, 2022 (JP) 2022-054506

(51) Int. Cl. A61B 8/08; A61B 8/00; A61B 8/12

(52) U.S. Cl. CPC A61B 8/085; A61B 8/12; A61B 8/463; A61B 8/5207

(57) ABSTRACT

An image processing apparatus includes a processor. The processor detects a first image region and a second image region from a medical image obtained by capturing an image of an observation target region including a site of a human body and a lesion, the first image region indicating the site, the second image region indicating the lesion. The processor causes a display apparatus to display a result of detection of the first image region and the second image region in a display mode in accordance with a positional relationship between the first image region and the second image region.

### CROSS-REFERENCE TO RELATED APPLICATIONS

This application is a continuation application of International Application No. PCT/JP2023/004985, filed Feb. 14, 2023, the disclosure of which is incorporated herein by reference in its entirety. Further, this application claims priority from Japanese Patent Application No. 2022-054506, filed Mar. 29, 2022, the disclosure of which is incorporated herein by reference in its entirety.

### BACKGROUND

### 1. Technical Field

The technology of the present disclosure relates to an image processing apparatus, a medical diagnostic apparatus, an ultrasonic endoscope apparatus, an image processing method, and a program.

### 2. Related Art

JP2021-100555A discloses a medical image processing apparatus having at least one processor. In the medical image processing apparatus described in JP2021-100555A, the at least one processor acquires a medical image, acquires site information indicating a site in a subject human body captured in the medical image, detects a lesion from the medical image to acquire lesion type information indicating a type of the lesion, determines whether the site information and the lesion type information are consistent with each other, and determines a notification manner of the site information and the lesion type information based on a result of the determination.

### SUMMARY

An embodiment according to the technology of the present disclosure provides an image processing apparatus, a medical diagnostic apparatus, an ultrasonic endoscope apparatus, an image processing method, and a program by which a user or the like can grasp a lesion with high accuracy.

A first aspect according to the technology of the present disclosure is an image processing apparatus including a processor, in which the processor is configured to: detect a first image region and a second image region from a medical image obtained by capturing an image of an observation target region including a site of a human body and a lesion, the first image region indicating the site, the second image region indicating the lesion; and cause a display apparatus to display a result of detection of the first image region and the second image region in a display mode in accordance with a positional relationship between the first image region and the second image region.

A second aspect according to the technology of the present disclosure is the image processing apparatus according to the first aspect, in which the display mode is determined in accordance with the site, the lesion, and the positional relationship.

A third aspect according to the technology of the present disclosure is the image processing apparatus according to the first or second aspect, in which the display mode is determined in accordance with the positional relationship and consistency between the site and the lesion.

A fourth aspect according to the technology of the present disclosure is the image processing apparatus according to any one of the first to third aspects, in which the display mode for the first image region differs depending on the site, the lesion, and the positional relationship, and the display mode for the second image region is a mode in which the second image region is displayed on the display apparatus.

A fifth aspect according to the technology of the present disclosure is the image processing apparatus according to the fourth aspect, in which, if the site and the lesion are not consistent with each other, the display mode for the first image region is a mode in which the first image region is not displayed on the display apparatus, and the display mode for the second image region is a mode in which the second image region is displayed on the display apparatus.

A sixth aspect according to the technology of the present disclosure is the image processing apparatus according to the fourth or fifth aspect, in which, if the site and the lesion are consistent with each other, the display mode for the first image region is a mode in which the first image region is displayed on the display apparatus and which is determined in accordance with the positional relationship, and the display mode for the second image region is a mode in which the second image region is displayed on the display apparatus.

A seventh aspect according to the technology of the present disclosure is the image processing apparatus according to any one of the first to sixth aspects, in which the positional relationship is defined by an overlapping degree or a distance between the first image region and the second image region.

An eighth aspect according to the technology of the present disclosure is the image processing apparatus according to the seventh aspect, in which, if the positional relationship is defined by the overlapping degree and the overlapping degree is greater than or equal to a first degree, the display mode is a mode in which the second image region is displayed so as to be identifiable in the medical image.

A ninth aspect according to the technology of the present disclosure is the image processing apparatus according the seventh aspect, in which, if the positional relationship is defined by the overlapping degree and the overlapping degree is greater than or equal to a first degree, the display mode is a mode in which the second image region is displayed so as to be identifiable in the medical image and the first image region is displayed so as to be comparable with the second image region.

A tenth aspect according to the technology of the present disclosure is the image processing apparatus according to any one of the first to ninth aspects, in which the processor is configured to acquire a first certainty factor and a second certainty factor, the first certainty factor being a certainty factor for the result of detection of the first image region, the second certainty factor being a certainty factor for the result of detection of the second image region, and the display mode is determined in accordance with the first certainty factor, the second certainty factor, and the positional relationship.

An eleventh aspect according to the technology of the present disclosure is the image processing apparatus according to the tenth aspect, in which the display mode is determined in accordance with a magnitude relationship between the first certainty factor and the second certainty factor and the positional relationship.

A twelfth aspect according to the technology of the present disclosure is the image processing apparatus according to any one of the first to eleventh aspects, in which the display mode is determined in accordance with a plurality of the positional relationships, and the plurality of the positional relationships are positional relationships between a plurality of the first image regions for a plurality of types of the sites and the second image region.

A thirteenth aspect according to the technology of the present disclosure is the image processing apparatus according to the twelfth aspect, in which the display mode for each of the plurality of the first image regions differs depending on a corresponding one of the plurality of the positional relationships.

A fourteenth aspect according to the technology of the present disclosure is the image processing apparatus according to the twelfth or thirteenth aspect, in which the display mode for each of the plurality of the first image regions differs depending on a first image region positional relationship between the plurality of the first image regions.

A fifteenth aspect according to the technology of the present disclosure is the image processing apparatus according to any one of the first to fourteenth aspects, in which the medical image is an image defined by a plurality of frames, the processor is configured to detect the first image region and the second image region for each of the frames, and the display mode is determined for each of the frames.

A sixteenth aspect according to the technology of the present disclosure is the image processing apparatus according to the fifteenth aspect, in which the processor is configured to: based on a correspondence relationship between a plurality of types of the sites and a lesion corresponding to each of the sites, determine whether a combination of the first image region and the second image region is correct for each of the frames; and based on the display mode corresponding to one of the frames used as a determination target if it is determined that the combination of the first image region and the second image region is correct, correct the display mode corresponding to one of the frames used as a determination target if it is determined that the combination of the first image region and the second image region is not correct.

A seventeenth aspect according to the technology of the present disclosure is a medical diagnostic apparatus including: the image processing apparatus according to any one of the first to sixteenth aspects; and an imaging apparatus configured to capture an image of the observation target region.

An eighteenth aspect according to the technology of the present disclosure is an ultrasonic endoscope apparatus including: the image processing apparatus according to any one of the first to sixteenth aspects; and an ultrasound apparatus configured to acquire an ultrasound image as the medical image.

A nineteenth aspect according to the technology of the present disclosure is an image processing method including: detecting a first image region and a second image region from a medical image obtained by imaging an observation target region including a site of a human body and a lesion, the first image region indicating the site, the second image region indicating the lesion; and causing a display apparatus to display a result of detection of the first image region and the second image region in a display mode in accordance with a positional relationship between the first image region and the second image region.

A twentieth aspect according to the technology of the present disclosure is a program for causing a computer to execute a process including: detecting a first image region and a second image region from a medical image obtained by imaging an observation target region including a site of a human body and a lesion, the first image region indicating the site, the second image region indicating the lesion; and causing a display apparatus to display a result of detection of the first image region and the second image region in a display mode in accordance with a positional relationship between the first image region and the second image region.

A twenty first aspect according to the technology of the present disclosure is an image processing apparatus including a processor, in which the processor is configured to: detect a first image region and a second image region from a medical image obtained by imaging an observation target region including a site of a human body and a lesion, the first image region indicating the site, the second image region indicating the lesion; and determine certainty of the second image region in accordance with a positional relationship between the first image region and the second image region.

A twenty second aspect according to the technology of the present disclosure is the image processing apparatus according to the twenty first aspect, in which the processor is configured to determine the certainty in accordance with the positional relationship and a relationship between a first certainty factor and a second certainty factor, the first certainty factor being a certainty factor for a result of detection of the first image region, the second certainty factor being a certainty factor for a result of detection of the second image region.

A twenty third aspect according to the technology of the present disclosure is the image processing apparatus according to the twenty second aspect, in which the processor is configured to determine that the second image region is certain if the positional relationship is a preset positional relationship, the first image region and the second image region are not consistent with each other, and the relationship between the first certainty factor and the second certainty factor is a preset certainty factor relationship.

A twenty fourth aspect according to the technology of the present disclosure is the image processing apparatus according to the twenty first aspect, in which the processor is configured to determine that the second image region is certain if the positional relationship is a preset positional relationship and the first image region and the second image region are consistent with each other.

A twenty fifth aspect according to the technology of the present disclosure is the image processing apparatus according to any one of the twenty first to twenty third aspects, in which the processor is configured to determine certainty of the first image region.

A twenty sixth aspect according to the technology of the present disclosure is the image processing apparatus according to any one of the twenty first to twenty fifth aspects, in which the processor is configured to: cause a display apparatus to display the medical image; and cause a display to display information indicating that the lesion is detected if it is determined that the second image region is certain.

A twenty seventh aspect according to the technology of the present disclosure is the image processing apparatus according to the twenty sixth aspect, in which a position at which the information indicating that the lesion is detected is displayed is a region corresponding to the second image region in a display region in which the medical image is displayed.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an ultrasonic endoscope system is used;

FIG. 2 is a conceptual diagram illustrating an example of an overall configuration of the ultrasonic endoscope system;

FIG. 3 is a conceptual diagram illustrating an example of an aspect in which an insertion unit of an ultrasonic endoscope is inserted into a stomach of an examinee;

FIG. 4 is a block diagram illustrating an example of a hardware configuration of an endoscope processing apparatus;

FIG. 5 is a block diagram illustrating an example of a hardware configuration of an ultrasound processing apparatus;

FIG. 6 is a block diagram illustrating an example of a hardware configuration of a display control apparatus;

FIG. 7 is a block diagram illustrating an example of functions of main parts of a processor of the display control apparatus;

FIG. 8 is a conceptual diagram illustrating an example of processing details of an acquisition unit;

FIG. 9 is a conceptual diagram illustrating an example of processing details of the acquisition unit, a detection unit, and a determination unit;

FIG. 10 is a conceptual diagram illustrating an example of processing details of the acquisition unit, the determination unit, and a control unit;

FIG. 11 is a conceptual diagram illustrating an example of processing details of the acquisition unit, the determination unit, and a positional relationship identification unit;

FIG. 12 is a conceptual diagram illustrating an example of processing details of the acquisition unit, the detection unit, the positional relationship identification unit, and the control unit in a case where an overlapping degree is less than a preset overlapping degree;

FIG. 13 is a conceptual diagram illustrating an example of processing details of the acquisition unit, the detection unit, the positional relationship identification unit, and the control unit in a case where the overlapping degree is greater than or equal to the preset overlapping degree;

FIG. 14 is a flowchart illustrating an example of a flow of a display control process;

FIG. 15 is a conceptual diagram illustrating an example of processing details of a first modification;

FIG. 16 is a conceptual diagram illustrating an example of processing details of a second modification;

FIG. 17 is a conceptual diagram illustrating an example of processing details of a third modification;

FIG. 18 is a conceptual diagram illustrating an example of processing details of the detection unit and the determination unit according to a fourth modification;

FIG. 19 is a conceptual diagram illustrating an example of processing details of the acquisition unit, the determination unit, the positional relationship identification unit, and the control unit in a case where a combination of a site region and a lesion region is not correct and the overlapping degree is less than the preset overlapping degree;

FIG. 20 is a conceptual diagram illustrating an example of processing details of the acquisition unit, the determination unit, the positional relationship identification unit, and the control unit in a case where the combination of the site region and the lesion region is not correct and the overlapping degree is greater than or equal to the preset overlapping degree;

FIG. 21 is a conceptual diagram illustrating an example of processing details of the acquisition unit, the determination unit, the positional relationship identification unit, and the control unit in a case where the combination of the site region and the lesion region is not correct and a second certainty factor is less than or equal to a first certainty factor;

FIG. 22 is a conceptual diagram illustrating an example of processing details of the acquisition unit, the determination unit, the positional relationship identification unit, and the control unit in a case where the combination of the site region and the lesion region is correct and the overlapping degree is less than the preset overlapping degree;

FIG. 23 is a conceptual diagram illustrating an example of processing details of the acquisition unit, the determination unit, the positional relationship identification unit, and the control unit in a case where the combination of the site region and the lesion region is correct and the overlapping degree is greater than or equal to the preset overlapping degree;

FIG. 24 is a conceptual diagram illustrating an example of processing details of the acquisition unit, the determination unit, the positional relationship identification unit, and the control unit in a case where the combination of the site region and the lesion region is correct and the second certainty factor is less than or equal to the first certainty factor;

FIG. 25A is a flowchart illustrating an example of a flow of a display control process according to the fourth modification;

FIG. 25B is a continuation of the flowchart illustrated in FIG. 25A;

FIG. 26 is a conceptual diagram illustrating an example of processing details of the detection unit and the determination unit according to a fifth modification;

FIG. 27A is a flowchart illustrating an example of a flow of a display control process according to the fifth modification;

FIG. 27B is a continuation of the flowchart illustrated in FIG. 27A;

FIG. 28 is a conceptual diagram illustrating an example of an ultrasound image of a related art and an example of an ultrasound image subjected to the display control process according to the fifth modification;

FIG. 29 is a conceptual diagram illustrating an example of an ultrasound image of a related art and an example of an ultrasound image subjected to a display control process according to a sixth modification;

FIG. 30A is a flowchart illustrating an example of a flow of a display control process according to a seventh modification;

FIG. 30B is a continuation of the flowchart illustrated in FIG. 30A;

FIG. 31A is a flowchart illustrating an example of a flow of a display control process according to an eighth modification;

FIG. 31B is a continuation of the flowchart illustrated in FIG. 31A;

FIG. 32 is a conceptual diagram illustrating an example of processing details of a control unit according to a ninth modification;

FIG. 33A is a flowchart illustrating an example of a flow of a display control process according to a tenth modification; and

FIG. 33B is a continuation of the flowchart illustrated in FIG. 33A.

### DETAILED DESCRIPTION

Hereinafter, an example of an embodiment of an image processing apparatus, a medical diagnostic apparatus, an ultrasonic endoscope apparatus, an image processing method, and a program according to the technology of the present disclosure will be described with reference to the accompanying drawings.

First of all, terms used in the following description will be described.

CPU is an abbreviation for “Central Processing Unit”. GPU is an abbreviation for “Graphics Processing Unit”. RAM is an abbreviation for “Random Access Memory”. NVM is an abbreviation for “Non-volatile memory”. EEPROM is an abbreviation for “Electrically Erasable Programmable Read-Only Memory”. ASIC is an abbreviation for “Application Specific Integrated Circuit”. PLD is an abbreviation for “Programmable Logic Device”. FPGA is an abbreviation for “Field-Programmable Gate Array”. SoC is an abbreviation for “System-on-a-chip”. SSD is an abbreviation for “Solid State Drive”. USB is an abbreviation for “Universal Serial Bus”. HDD is an abbreviation for “Hard Disk Drive”. EL is an abbreviation for “Electro-Luminescence”. CMOS is an abbreviation for “Complementary Metal Oxide Semiconductor”. CCD is an abbreviation for “Charge Coupled Device”. CT is an abbreviation for “Computed Tomography”. MRI is an abbreviation for “Magnetic Resonance Imaging”. AI is an abbreviation for “Artificial Intelligence”. FIFO is an abbreviation for “First In First Out”. FPC is an abbreviation for “Flexible Printed Circuit”. IoU is an abbreviation for “Intersection over Union”.

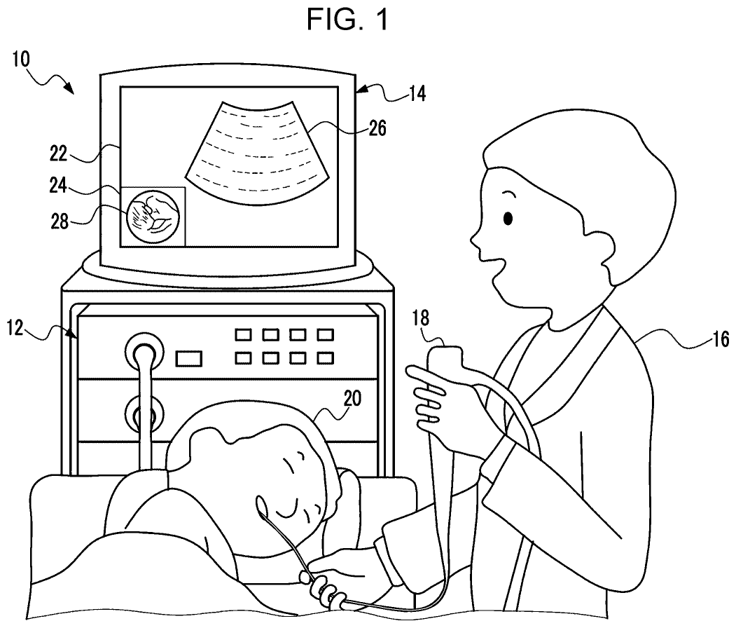

As illustrated in FIG. 1 as an example, an ultrasonic endoscope system 10 includes an ultrasonic endoscope apparatus 12 and a display apparatus 14. The ultrasonic endoscope apparatus 12 is used by a medical person (hereinafter referred to as a “user”) such as a doctor 16, a nurse, and/or a technician. The ultrasonic endoscope apparatus 12 includes an ultrasonic endoscope 18 and is an apparatus for performing medical care inside a body of an examinee 20 (e.g., a patient) through the ultrasonic endoscope 18. The ultrasonic endoscope apparatus 12 is an example of a “medical diagnostic apparatus” and an “ultrasonic endoscope apparatus” according to the technology of the present disclosure. The ultrasonic endoscope 18 is an example of an “imaging apparatus” according to the technology of the present disclosure.

The doctor 16 captures an image of the examinee 20, and thus, the ultrasonic endoscope 18 acquires and outputs an image indicating an internal body state. The example illustrated in FIG. 1 illustrates a state in which the ultrasonic endoscope 18 is inserted into a body cavity from a mouth of the examinee 20. Note that, although the ultrasonic endoscope 18 is inserted into the body cavity from the mouth of the examinee 20 in the example illustrated in FIG. 1, this is merely an example, and the ultrasonic endoscope 18 may be inserted into the body cavity from a nostril, an anus, a perforation, or the like of the examinee 20.

The display apparatus 14 displays various kinds of information including an image. An example of the display apparatus 14 is a liquid crystal display, an EL display, or the like. A plurality of screens are displayed side by side on the display apparatus 14. In the example illustrated in FIG. 1, a first screen 22 and a second screen 24 are illustrated as examples of the plurality of screens.

Different types of images obtained by the ultrasonic endoscope apparatus 12 are displayed on the first screen 22 and the second screen 24. An ultrasound moving image 26 is displayed on the first screen 22. The ultrasound moving image 26 is a moving image generated based on an echo obtained by emitting ultrasound toward an observation target region in the body of the examinee 20 and reflecting the ultrasound on the observation target region. The ultrasound moving image 26 is displayed on the first screen 22 by a live view method. Although the live view method is given as an example herein, this is merely an example, and another display method such as a post view method may be used. An example of the observation target region irradiated with the ultrasound is a region including an organ and a lesion of the examinee 20.

The observation target region irradiated with the ultrasound herein is an example of an “observation target region” according to the technology of the present disclosure. In addition, the organ and the lesion of the examinee are examples of a “site and a lesion of a human body” according to the technology of the present disclosure. In addition, the ultrasound moving image 26 (i.e., the moving image generated based on the echo obtained by reflecting the ultrasound on the observation target region) is an example of a “medical image obtained by capturing an image of the observation target region” according to the technology of the present disclosure.

An endoscopic moving image 28 is displayed on the second screen 24. An example of the endoscopic moving image 28 is a moving image generated by capturing an image of visible light, near-infrared light, or the like. The endoscopic moving image 28 is displayed on the second screen 24 by a live view method. Note that, although the endoscopic moving image 28 is illustrated together with the ultrasound moving image 26 in this embodiment, this is merely an example, and the technology of the present disclosure is established without the endoscopic moving image 28.

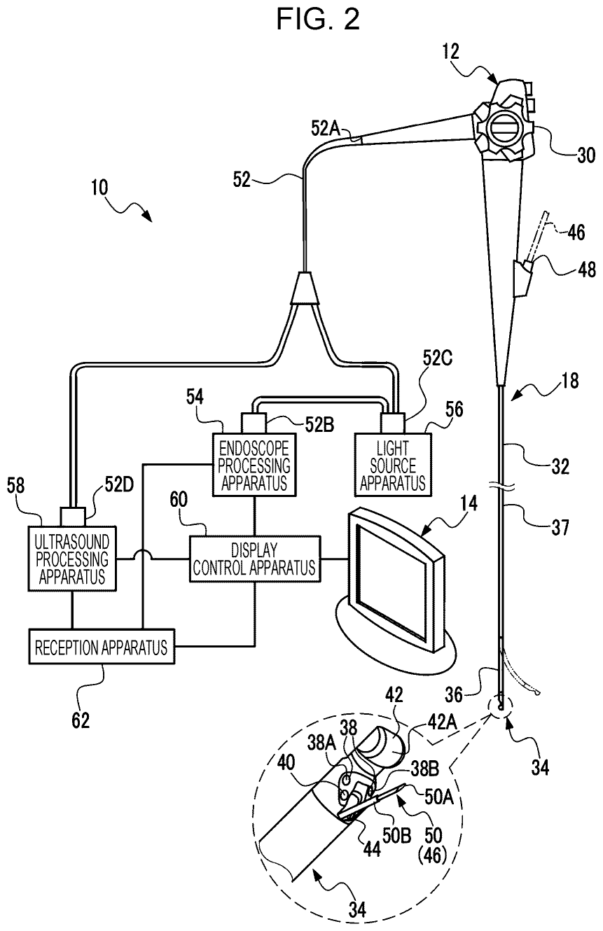

As illustrated in FIG. 2 as an example, the ultrasonic endoscope 18 includes an operating unit 30 and an insertion unit 32. The insertion unit 32 is formed in a tubular shape. The insertion unit 32 has a tip part 34, a bending part 36, and a soft part 37. The tip part 34, the bending part 36, and the soft part 37 are arranged in the order of the tip part 34, the bending part 36, and the soft part 37 from the distal end side to the proximal end side of the insertion unit 32. The soft part 37 is formed of an elongated flexible material and connects the operating unit 30 and the bending part 36 to each other. The bending part 36 is partly bent or rotated around the axis of the insertion unit 32 by operating the operating unit 30. As a result, the insertion unit 32 is fed to the back side in the body cavity while being curved and being rotated around the axis of the insertion unit 32 in accordance with the shape of the body cavity (e.g., the shape of a digestive tract such as an esophagus, a stomach, a duodenum, a small intestine, or a large intestine, or the shape of a bronchial tube).

The tip part 34 is provided with an illumination apparatus 38, an endoscope 40, an ultrasound probe 42, and a treatment tool opening 44. The illumination apparatus 38 has an illumination window 38A and an illumination window 38B. The illumination apparatus 38 emits light (e.g., white light formed of three primary color light or near-infrared light) through the illumination window 38A and the illumination window 38B. The endoscope 40 captures an image of the inside of the body by an optical method. An example of the endoscope 40 is a CMOS camera. The CMOS camera is merely an example, and another type of camera such as a CCD camera may be used.

The ultrasound probe 42 is provided on the distal end side of the tip part 34. An outer surface 42A of the ultrasound probe 42 is convexly curved outward from the proximal end side toward the distal end side of the ultrasound probe 42. The ultrasound probe 42 transmits ultrasound through the outer surface 42A and receives, through the outer surface 42A, an echo obtained by the transmitted ultrasound being reflected on the observation target region.

The treatment tool opening 44 is formed closer to the proximal end side of the tip part 34 than the ultrasound probe 42. This is an opening for projecting a treatment tool 46 from the tip part 34. A treatment tool insertion port 48 is formed in the operating unit 30, and the treatment tool 46 is inserted into the insertion unit 32 from the treatment tool insertion port 48. The treatment tool 46 passes through the insertion unit 32 and protrudes from the treatment tool opening 44 into the body. In the example illustrated in FIG. 2, as the treatment tool 46, a puncture needle 50 with a guide sheath protrudes from the treatment tool opening 44. The puncture needle 50 with the guide sheath has a puncture needle 50A and a guide sheath 50B. The puncture needle 50A passes through the guide sheath 50B and protrudes from the guide sheath 50B. Although the puncture needle 50 with the guide sheath is given as an example herein as the treatment tool 46, this is merely an example, and the treatment tool 46 may be grasping forceps, a scalpel, a snare, and/or the like. Note that the treatment tool opening 44 also functions as a suction port for sucking blood, body waste, and the like.

The ultrasonic endoscope apparatus 12 includes a universal cord 52, an endoscope processing apparatus 54, a light source apparatus 56, an ultrasound processing apparatus 58, and a display control apparatus 60. The universal cord 52 has a proximal end part 52A and first to third tip parts 52B to 52D. The proximal end part 52A is connected to the operating unit 30. The first tip part 52B is connected to the endoscope processing apparatus 54. The second tip part 52C is connected to the light source apparatus 56. The third tip part 52D is connected to the ultrasound processing apparatus 58.

The ultrasonic endoscope system 10 includes a reception apparatus 62. The reception apparatus 62 receives an instruction from a user. Examples of the reception apparatus 62 include an operation panel having a plurality of hard keys, a touch panel, and/or the like, a keyboard, a mouse, a trackball, a foot switch, a smart device, a microphone, and/or the like.

The reception apparatus 62 is connected to the endoscope processing apparatus 54. In accordance with an instruction received by the reception apparatus 62, the endoscope processing apparatus 54 transmits and receives various signals to and from the endoscope 40 and controls the light source apparatus 56. The endoscope processing apparatus 54 causes the endoscope 40 to capture an image, and acquires and outputs the endoscopic moving image 28 (see FIG. 1) from the endoscope 40. The light source apparatus 56 emits light under the control of the endoscope processing apparatus 54 and supplies the light to the illumination apparatus 38. The illumination apparatus 38 incorporates a light guide, and light supplied from the light source apparatus 56 is emitted from the illumination windows 38A and 38B via the light guide.

The reception apparatus 62 is connected to the ultrasound processing apparatus 58. The ultrasound processing apparatus 58 transmits and receives various signals to and from the ultrasound probe 42 in accordance with an instruction received by the reception apparatus 62. The ultrasound processing apparatus 58 causes the ultrasound probe 42 to transmit ultrasound, and generates and outputs the ultrasound moving image 26 based on an echo received by the ultrasound probe 42.

The display apparatus 14, the endoscope processing apparatus 54, the ultrasound processing apparatus 58, and the reception apparatus 62 are connected to the display control apparatus 60. The display control apparatus 60 controls the display apparatus 14 in accordance with an instruction received by the reception apparatus 62. The display control apparatus 60 acquires the endoscopic moving image 28 from the endoscope processing apparatus 54, and causes the display apparatus 14 to display the acquired endoscopic moving image 28 (see FIG. 1). In addition, the display control apparatus 60 acquires the ultrasound moving image 26 from the ultrasound processing apparatus 58, and causes the display apparatus 14 to display the acquired ultrasound moving image 26 (see FIG. 1).

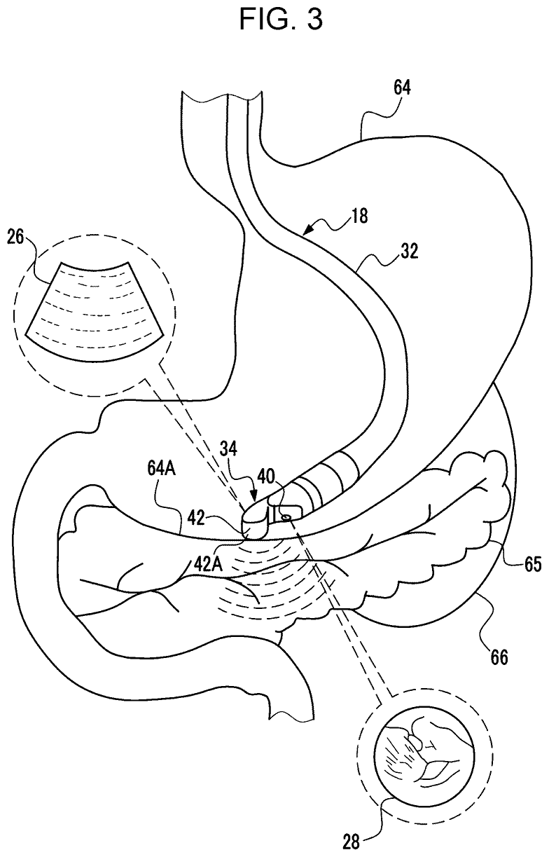

As illustrated in FIG. 3 as an example, the insertion unit 32 of the ultrasonic endoscope 18 is inserted into a stomach 64 of the examinee 20. The endoscope 40 captures an image of the inside of the stomach 64 at a preset frame rate (e.g., 30 frames/second or 60 frames/second) to generate a live view image indicating a state of the inside of the stomach 64 as the endoscopic moving image 28.

When the tip part 34 reaches a target position inside the stomach 64, the outer surface 42A of the ultrasound probe 42 comes into contact with an inner wall 64A of the stomach 64. In a state in which the outer surface 42A is in contact with the inner wall 64A, the ultrasound probe 42 transmits ultrasound through the outer surface 42A. The observation target region including organs such as a pancreas 65 and a kidney 66 and a lesion is irradiated with the ultrasound through the inner wall 64A. The echo obtained by the ultrasound being reflected on the observation target region is received by the ultrasound probe 42 via the outer surface 42A. Then, the echo received by the ultrasound probe 42 is imaged as a live view image indicating a state of the observation target region in accordance with the preset frame rate, and thus, the ultrasound moving image 26 is obtained.

Note that, although the example illustrated in FIG. 3 illustrates an example of a form in which the organs such as the pancreas 65 and the kidney 66 are irradiated with the ultrasound from the inside of the stomach 64 in order to detect a lesion of the pancreas 65, this is merely an example. For example, in order to detect a lesion of the pancreas 65, organs such as the pancreas 65 and the kidney 66 may be irradiated with the ultrasound from the inside of the duodenum.

In addition, although the example illustrated in FIG. 3 illustrates a state in which the ultrasonic endoscope 18 is inserted into the stomach 64, this is merely an example, and the ultrasonic endoscope 18 may be inserted into an organ such as an intestine and/or a bronchus.

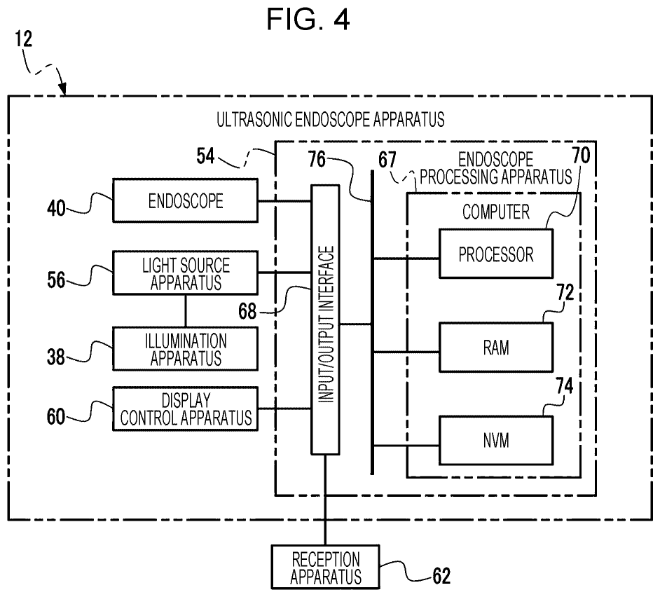

As illustrated in FIG. 4 as an example, the endoscope processing apparatus 54 includes a computer 67 and an input/output interface 68. The computer 67 includes a processor 70, a RAM 72, and an NVM 74. The input/output interface 68, the processor 70, the RAM 72, and the NVM 74 are connected to a bus 76.

For example, the processor 70 has a CPU and a GPU, and controls the entirety of the endoscope processing apparatus 54. The GPU operates under the control of the CPU and executes various kinds of graphic processing. Note that the processor 70 may be one or more CPUs in which GPU functions are integrated, or may be one or more CPUs in which GPU functions are not integrated.

The RAM 72 is a memory in which information is temporarily stored, and is used as a work memory by the processor 70. The NVM 74 is a non-volatile storage device that stores various programs, various parameters, and the like. An example of the NVM 74 is a flash memory (e.g., an EEPROM and/or an SSD). Note that the flash memory is merely an example, and may be another non-volatile storage device such as an HDD or may be a combination of two or more types of non-volatile storage devices.

The reception apparatus 62 is connected to the input/output interface 68, and the processor 70 acquires an instruction received by the reception apparatus 62 via the input/output interface 68 and executes processing in accordance with the acquired instruction. The endoscope 40 is also connected to the input/output interface 68. The processor 70 controls the endoscope 40 via the input/output interface 68, and acquires, via the input/output interface 68, the endoscopic moving image 28 obtained by capturing an image of the inside of the body of the examinee 20 with the endoscope 40. The light source apparatus 56 is also connected to the input/output interface 68. By controlling the light source apparatus 56 via the input/output interface 68, the processor 70 supplies light to the illumination apparatus 38 and adjusts the amount of light to be supplied to the illumination apparatus 38. The display control apparatus 60 is also connected to the input/output interface 68. The processor 70 transmits and receives various signals to and from the display control apparatus 60 via the input/output interface 68.

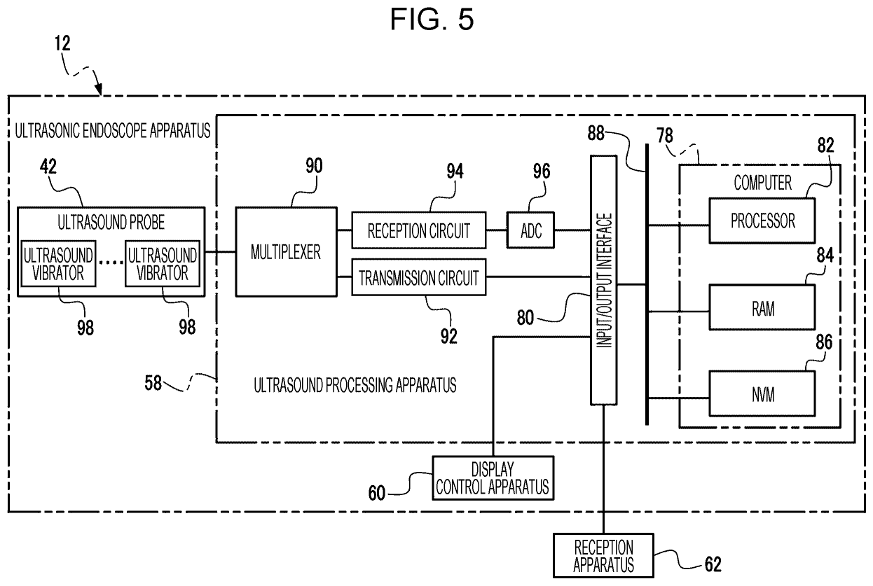

As illustrated in FIG. 5 as an example, the ultrasound processing apparatus 58 includes a computer 78 and an input/output interface 80. The computer 78 includes a processor 82, a RAM 84, and an NVM 86. The input/output interface 80, the processor 82, the RAM 84, and the NVM 86 are connected to a bus 88. The processor 82 controls the entirety of the ultrasound processing apparatus 58. Note that a plurality of hardware resources (the processor 82, the RAM 84, and the NVM 86) included in the computer 78 illustrated in FIG. 5 are of the same type as a plurality of hardware resources included in the computer 67 illustrated in FIG. 4, and thus, description thereof is omitted herein.

The reception apparatus 62 is connected to the input/output interface 80, and the processor 82 acquires an instruction received by the reception apparatus 62 via the input/output interface 80 and executes processing in accordance with the acquired instruction. The display control apparatus 60 is also connected to the input/output interface 80. The processor 82 transmits and receives various signals to and from the display control apparatus 60 via the input/output interface 80.

The ultrasound processing apparatus 58 includes a multiplexer 90, a transmission circuit 92, a reception circuit 94, and an analog-to-digital converter 96 (hereinafter referred to as “ADC 96”). The multiplexer 90 is connected to the ultrasound probe 42. An input terminal of the transmission circuit 92 is connected to the input/output interface 80, and an output terminal of the transmission circuit 92 is connected to the multiplexer 90. An input terminal of the ADC 96 is connected to an output terminal of the reception circuit 94, and an output terminal of the ADC 96 is connected to the input/output interface 80. An input terminal of the reception circuit 94 is connected to the multiplexer 90.

The ultrasound probe 42 includes a plurality of ultrasound vibrators 98. The ultrasound probe 42 is formed by laminating, from the inside to the outside of the ultrasound probe 42, a backing material layer, an ultrasound vibrator unit (i.e., a unit in which the plurality of ultrasound vibrators 98 are arranged in a one dimensional or two dimensional array), an acoustic matching layer, and an acoustic lens.

The backing material layer supports each ultrasound vibrator 98 included in the ultrasound vibrator unit from the back surface side. In addition, the backing material layer has a function of attenuating ultrasound propagating from the ultrasound vibrator 98 to the backing material layer side. The backing material is formed of a material having stiffness, such as hard rubber, and an ultrasound attenuating material (e.g., ferrite or ceramics) is added thereto as necessary.

The acoustic matching layer is superposed on the ultrasound vibrator unit and is provided to achieve acoustic impedance matching between the examinee 20 and the ultrasound vibrator 98. Since the acoustic matching layer is provided, it is possible to increase the transmittance of ultrasound. The material of the acoustic matching layer may be an organic material having an acoustic impedance value closer to that of the examinee 20 than that of a piezoelectric element included in the ultrasound vibrator 98. Examples of the material of the acoustic matching layer include an epoxy-based resin, silicone rubber, polyimide, polyethylene, and/or the like.

The acoustic lens is superposed on the acoustic matching layer and is a lens that converges ultrasound emitted from the ultrasound vibrator unit toward the observation target region. The acoustic lens is formed of a silicon-based resin, a liquid silicone rubber, a butadiene-based resin, a polyurethane-based resin, and/or the like, and powder of titanium oxide, alumina, silica, or the like is mixed therein as necessary.

Each of the plurality of ultrasound vibrators 98 is formed by disposing electrodes on both surfaces of the piezoelectric element. Examples of the piezoelectric element include barium titanate, lead zirconate titanate, and potassium niobate. The electrodes are formed of individual electrodes individually provided for the plurality of ultrasound vibrators 98 and a vibrator ground common to the plurality of ultrasound vibrators 98. The electrodes are electrically connected to the ultrasound processing apparatus 58 via an FPC and a coaxial cable.

The ultrasound probe 42 is a convex probe in which the plurality of ultrasound vibrators 98 are arranged in an arc shape. The plurality of ultrasound vibrators 98 are arranged along the outer surface 42A (see FIG. 2 and FIG. 3). That is, the plurality of ultrasound vibrators 98 are arranged at equal intervals in a convex curved shape along the axial direction of the tip part 34 (i.e., the longitudinal axis direction of the insertion unit 32). Thus, the ultrasound probe 42 radially transmits ultrasound by operating the plurality of ultrasound vibrators 98. Note that, although the convex probe is given as an example herein, this is merely an example, and a radial probe, a linear probe, a sector probe, or the like may be used. In addition, a scanning method of the ultrasound probe 42 is not particularly limited.

The transmission circuit 92 and the reception circuit 94 are electrically connected to each of the plurality of ultrasound vibrators 98 via the multiplexer 90. The multiplexer 90 selects at least one of the plurality of ultrasound vibrators 98 and opens a channel of a selected ultrasound vibrator, which is the selected ultrasound vibrator 98.

The transmission circuit 92 is controlled by the processor 82 via the input/output interface 80. Under the control of the processor 82, the transmission circuit 92 supplies a driving signal (e.g., a plurality of pulsed signals) for ultrasound transmission to the selected ultrasound vibrator. The driving signal is generated in accordance with transmission parameters set by the processor 82. The transmission parameters are the number of driving signals to be supplied to the selected ultrasound vibrator, a supply time of the driving signal, an amplitude of driving vibration, and the like.

The transmission circuit 92 causes the selected ultrasound vibrator to transmit ultrasound by supplying the driving signal to the selected ultrasound vibrator. That is, when the driving signal is supplied to the electrodes included in the selected ultrasound vibrator, the piezoelectric element included in the selected ultrasound vibrator expands and contracts, and the selected ultrasound vibrator vibrates. As a result, pulsed ultrasound is output from the selected ultrasound vibrator. The output intensity of the selected ultrasound vibrator is defined by the amplitude of the ultrasound (i.e., the magnitude of the sound pressure of the ultrasound) output from the selected ultrasound vibrator.

An echo obtained by transmitting the ultrasound and reflecting it on the observation target region is received by the ultrasound vibrator 98. The ultrasound vibrator 98 outputs an electric signal indicating the received echo to the reception circuit 94 via the multiplexer 90. Specifically, the piezoelectric element included in the ultrasound vibrator 98 outputs an electric signal. The magnitude (i.e., voltage value) of the electric signal output from the ultrasound vibrator 98 corresponds to the reception sensitivity of the ultrasound vibrator 98. The reception sensitivity of the ultrasound vibrator 98 is defined as a ratio of the amplitude of the electric signal output by the ultrasound vibrator 98 receiving the ultrasound to the amplitude of the ultrasound transmitted by the ultrasound vibrator 98. The reception circuit 94 receives the electric signal from the ultrasound vibrator 98, amplifies the received electric signal, and outputs the amplified electric signal to the ADC 96. The ADC 96 digitizes the electric signal input from the reception circuit 94. The processor 82 acquires the electric signal digitized by the ADC 96 and generates the ultrasound moving image 26 (see FIG. 1 and FIG. 3) based on the acquired electric signal, thereby acquiring the ultrasound moving image 26.

Note that in this embodiment, a combination of the ultrasound probe 42 and the ultrasound processing apparatus 58 is an example of an “imaging apparatus” according to the technology of the present disclosure. In addition, in this embodiment, a combination of the ultrasound probe 42 and the ultrasound processing apparatus 58 is also an example of an “ultrasound apparatus” according to the technology of the present disclosure.

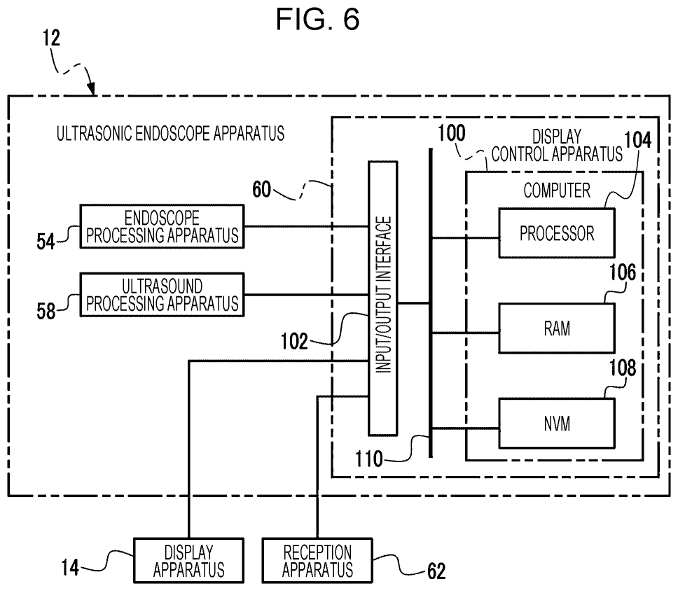

As illustrated in FIG. 6 as an example, the display control apparatus 60 includes a computer 100 and an input/output interface 102. The computer 100 includes a processor 104, a RAM 106, and an NVM 108. The input/output interface 102, the processor 104, the RAM 106, and the NVM 108 are connected to a bus 110.

The display control apparatus 60 herein is an example of an “image processing apparatus” according to the technology of the present disclosure. In addition, the computer 100 is an example of a “computer” according to the technology of the present disclosure. In addition, the processor 104 is an example of a “processor” according to the technology of the present disclosure.

The processor 104 controls the entirety of the display control apparatus 60. Note that a plurality of hardware resources (the processor 104, the RAM 106, and the NVM 108) included in the computer 100 illustrated in FIG. 6 are of the same type as the plurality of hardware resources included in the computer 67 illustrated in FIG. 4, and thus, description thereof is omitted herein.

The reception apparatus 62 is connected to the input/output interface 102, and the processor 104 acquires an instruction received by the reception apparatus 62 via the input/output interface 102 and executes processing in accordance with the acquired instruction. In addition, the display apparatus 14 is connected to the input/output interface 102. In addition, the endoscope processing apparatus 54 is connected to the input/output interface 102, and the processor 104 transmits and receives various signals to and from the processor 70 of the endoscope processing apparatus 54 via the input/output interface 102. In addition, the ultrasound processing apparatus 58 is connected to the input/output interface 102, and the processor 104 transmits and receives various signals to and from the processor 82 of the ultrasound processing apparatus 58 via the input/output interface 102.

The display apparatus 14 is connected to the input/output interface 102, and the processor 104 causes the display apparatus 14 to display various kinds of information by controlling the display apparatus 14 via the input/output interface 102. For example, the processor 104 acquires the endoscopic moving image 28 (see FIG. 1 and FIG. 3) from the endoscope processing apparatus 54, acquires the ultrasound moving image 26 (see FIG. 1 and FIG. 3) from the ultrasound processing apparatus 58, and causes the display apparatus 14 to display the ultrasound moving image 26 and the endoscopic moving image 28.

The ultrasonic endoscope 18 irradiates the inside of the body of the examinee 20 with ultrasound and images an echo obtained by the ultrasound being reflected on the observation target region as the ultrasound moving image 26, and thus, it is possible to detect a lesion included in the observation target region while suppressing a physical load applied to the examinee 20. However, the ultrasound moving image 26 has lower visibility than a visible light image (e.g., the endoscopic moving image 28), and there is a possibility that a lesion may be missed or diagnostic results may vary according to the doctor 16. Thus, in recent years, in order to suppress missing of a lesion and/or variation in diagnosis results according to the doctor 16, AI image recognition processing has been used.

However, even if a lesion is detected by the AI image recognition processing, a plurality of organs may be captured together with the lesion in the ultrasound moving image 26, and it is difficult to identify the organ to which the lesion belongs among the plurality of organs. Furthermore, if a plurality of organs are captured in an overlapping manner in the ultrasound moving image 26, it is more difficult to identify the organ to which the lesion belongs.

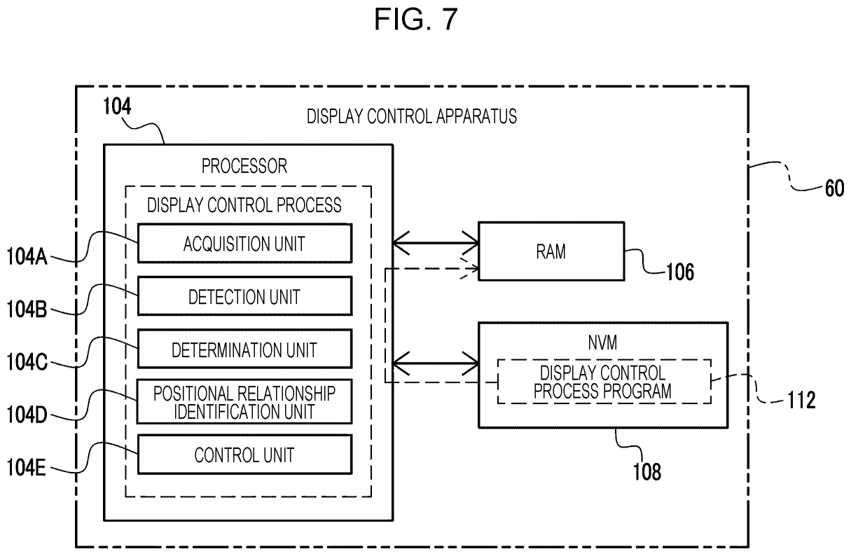

In view of such circumstances, in this embodiment, as illustrated in FIG. 7 as an example, the processor 104 performs a display control process in the display control apparatus 60. The NVM 108 stores a display control process program 112. The display control process program 112 is an example of a “program” according to the technology of the present disclosure. The processor 104 performs the display control process by reading the display control process program 112 from the NVM 108 and executing the read display control process program 112 on the RAM 106. The display control process is implemented by the processor 104 operating as an acquisition unit 104A, a detection unit 104B, a determination unit 104C, a positional relationship identification unit 104D, and a control unit 104E in accordance with the display control process program 112.

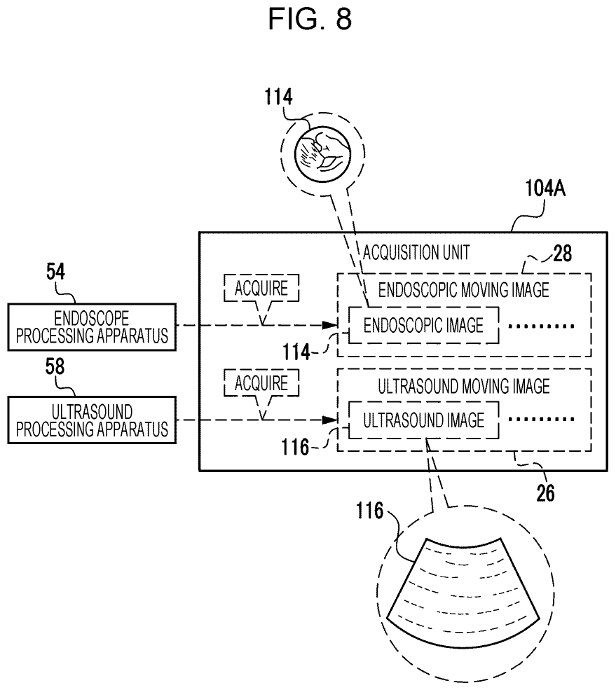

As illustrated in FIG. 8 as an example, the acquisition unit 104A acquires the endoscopic moving image 28 from the endoscope processing apparatus 54. The endoscopic moving image 28 is an image defined by a plurality of frames. That is, the endoscopic moving image 28 includes a plurality of endoscopic images 114 obtained as a plurality of time-series frames by the endoscope processing apparatus 54 in accordance with a preset frame rate. In addition, the acquisition unit 104A acquires the ultrasound moving image 26 from the ultrasound processing apparatus 58. The ultrasound moving image 26 is an image defined by a plurality of frames. That is, the ultrasound moving image 26 includes a plurality of ultrasound images 116 obtained as a plurality of time-series frames by the ultrasound processing apparatus 58 in accordance with a preset frame rate.

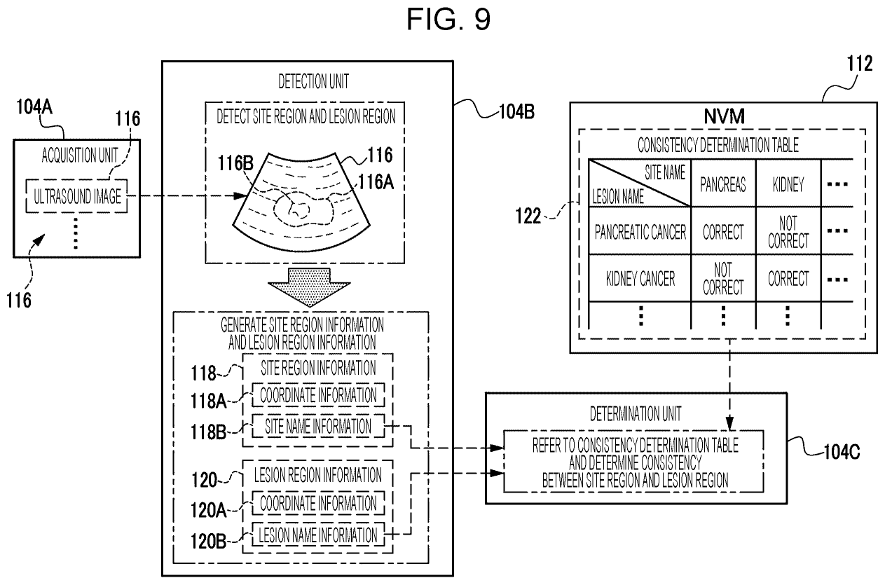

As illustrated in FIG. 9 as an example, the detection unit 104B detects a site region 116A and a lesion region 116B from an ultrasound image 116 acquired by the acquisition unit 104A. The site region 116A is an image region indicating an organ (e.g., a pancreas) captured in the ultrasound image 116. The lesion region 116B is an image region indicating a lesion (e.g., a tumor) captured in the ultrasound image 116. The site region 116A is an example of a “first image region” according to the technology of the present disclosure, and the lesion region 116B is an example of a “second image region” according to the technology of the present disclosure.

The detection unit 104B detects the site region 116A and the lesion region 116B for each frame (i.e., for each of the plurality of ultrasound images 116 included in the ultrasound moving image 26). Then, the display control process is performed for each frame. Hereinafter, in order to facilitate understanding of the technology of the present disclosure, a case where the display control process is performed on one frame will be described.

The detection unit 104B performs the AI image recognition processing on the ultrasound image 116 to detect the site region 116A and the lesion region 116B from the ultrasound image 116. Although the AI image recognition processing is given as an example herein, this is merely an example, and the site region 116A and the lesion region 116B may be detected by image recognition processing by a template matching method instead of the AI image recognition processing. In addition, the detection unit 104B may use both the AI image recognition processing and the image recognition processing by the template matching method.

The detection unit 104B generates site region information 118 and lesion region information 120. The site region information 118 is information related to the site region 116A detected by the detection unit 104B. The site region information 118 includes coordinate information 118A and site name information 118B. The coordinate information 118A is information including coordinates (e.g., two dimensional coordinates) by which the position of the site region 116A (e.g., the position of the contour of the site region 116A) in the ultrasound image 116 can be identified. The site name information 118B is information (e.g., information indicating the name of the organ itself or an identifier by which the type of the organ can be uniquely identified) by which the name of the site (i.e., the type of the site) indicated by the site region 116A detected by the detection unit 104B can be identified.

The lesion region information 120 is information related to the lesion region 116B detected by the detection unit 104B. The lesion region information 120 includes coordinate information 120A and lesion name information 120B. The coordinate information 120A is information including coordinates (e.g., two dimensional coordinates) by which the position of the lesion region 116B (e.g., the position of the contour of the lesion region 116B) in the ultrasound image 116 can be identified. The lesion name information 120B is information (e.g., information indicating the name of the lesion or an identifier by which the type of the lesion can be uniquely identified) by which the name of the lesion (i.e., the type of the lesion) indicated by the lesion region 116B detected by the detection unit 104B can be identified.

The NVM 108 stores a consistency determination table 122. In the consistency determination table 122, a plurality of pieces of site name information 118B and a plurality of pieces of lesion name information 120B are associated with each other in a one-to-one manner. That is, the consistency determination table 122 defines the name of the site identified from the site name information 118B and the name of the lesion identified from the lesion name information 120B. In other words, the consistency determination table 122 defines a correct combination of a site and a lesion. In the example illustrated in FIG. 9, as an example of some correct combinations of sites and lesions, a combination of a pancreas and pancreatic cancer and a combination of a kidney and kidney cancer are illustrated. The consistency determination table 122 is an example of a “correspondence relationship” according to the technology of the present disclosure.

The determination unit 104C acquires the site name information 118B and the lesion name information 120B from the site region information 118. In addition, the determination unit 104C refers to the consistency determination table 122 stored in the NVM 108 and determines the consistency of the combination of the site name information 118B and the lesion name information 120B to determine the consistency between the site region 116A and the lesion region 116B (in other words, whether the combination of the site and the lesion is correct). That is, the determination unit 104C refers to the consistency determination table 122 and determines whether the combination of the name of the site identified from the site name information 118B and the name of the lesion identified from the lesion name information 120B is correct or not. Thus, the determination unit 104C determines whether the combination of the site region 116A and the lesion region 116B detected by the detection unit 104B is correct or not (i.e., whether the combination of the site region 116A and the lesion region 116B is consistent or not).

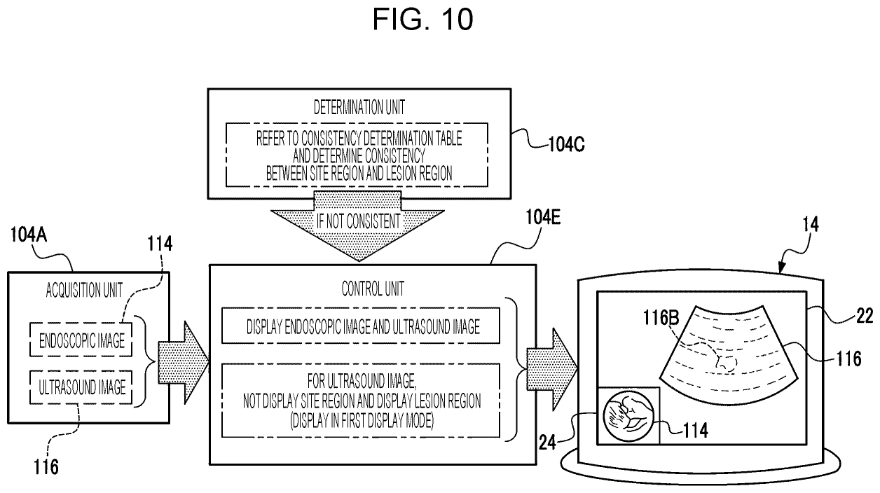

As illustrated in FIG. 10 as an example, the control unit 104E acquires an endoscopic image 114 and the ultrasound image 116 that is a determination target of the determination unit 104C, displays the acquired ultrasound image 116 on the first screen 22, and displays the acquired endoscopic image 114 on the second screen 24. In this case, if the determination unit 104C determines that the site region 116A and the lesion region 116B are not consistent with each other, the control unit 104E displays the ultrasound image 116 on the first screen 22 in a first display mode. The first display mode is a display mode in which the site region 116A is not displayed and the lesion region 116B is displayed. In the example illustrated in FIG. 10, in the ultrasound image 116 on the first screen 22, the site region 116A is not displayed, and the lesion region 116B is displayed.

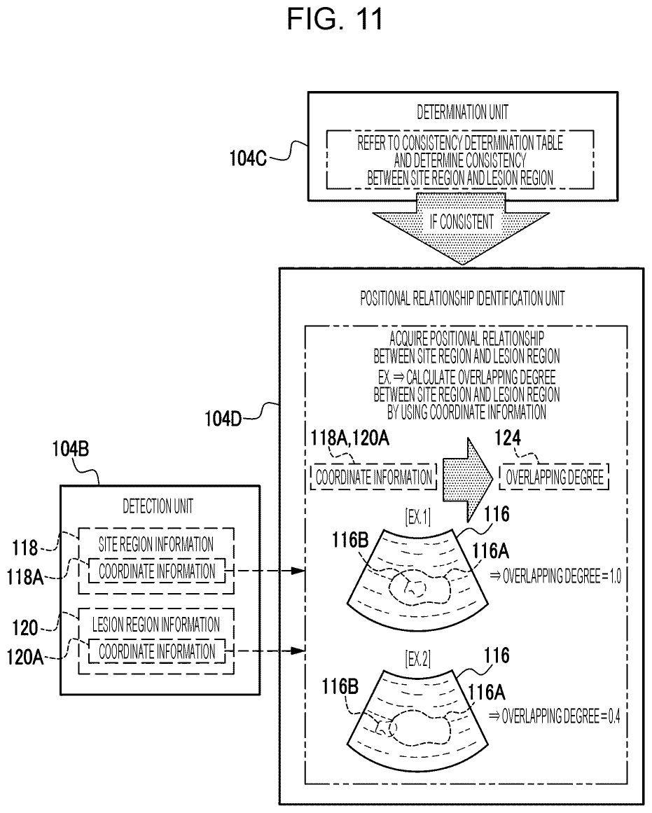

As illustrated in FIG. 11 as an example, if the determination unit 104C determines that the site region 116A and the lesion region 116B are consistent with each other, the positional relationship identification unit 104D acquires a positional relationship between the site region 116A and the lesion region 116B. As an example, the positional relationship between the site region 116A and the lesion region 116B is defined by an overlapping degree, which is a degree to which the site region 116A and the lesion region 116B overlap with each other. The positional relationship identification unit 104D acquires the coordinate information 118A from the site region information 118, acquires the coordinate information 120A from the lesion region information 120, and calculates an overlapping degree 124 between the site region 116A and the lesion region 116B by using the coordinate information 118A and 120A. An example of an index of the overlapping degree 124 is IoU. In this case, for example, the overlapping degree 124 is a ratio of the area of a region where the lesion region 116B and the site region 116A overlap with each other to the area of a region obtained by combining the lesion region 116B and the site region 116A. In the example illustrated in FIG. 11, a state in which the entirety of the lesion region 116B overlaps with the site region 116A is indicated as “overlapping degree=1.0”, and a state in which part of the lesion region 116B overlaps with the site region 116A is indicated as “overlapping degree=0.4”.

Note that, although IoU is given as an example herein, the technology of the present disclosure is not limited to this, and the overlapping degree 124 may be a ratio of the area of the region where the lesion region 116B and the site region 116A overlap with each other to the lesion region 116B.

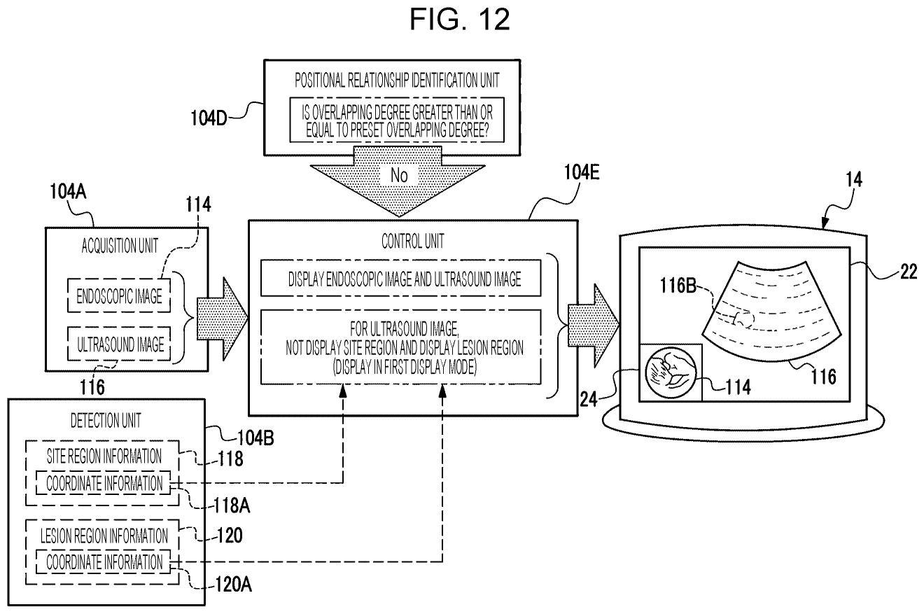

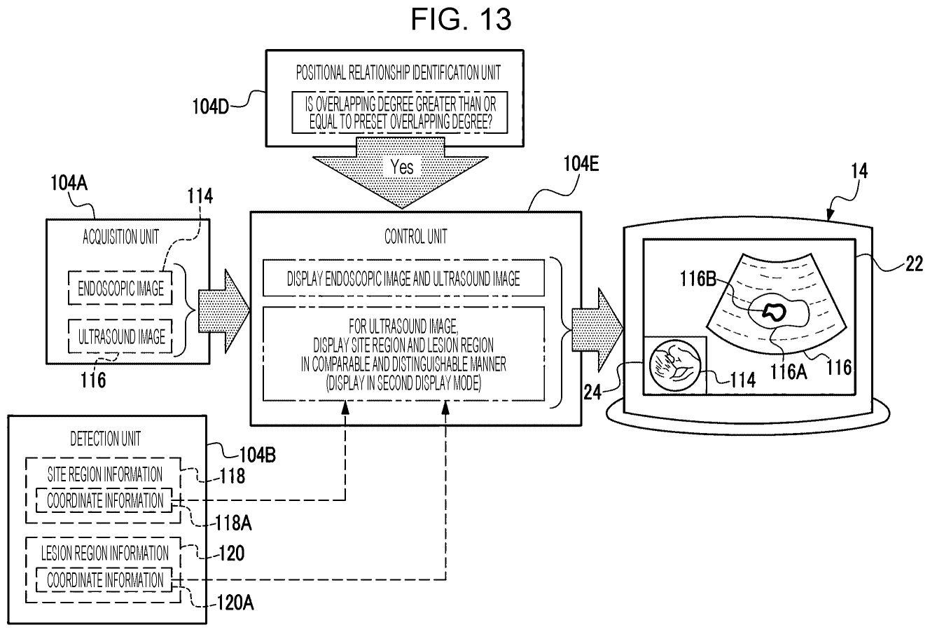

As illustrated in FIG. 12 and FIG. 13 as examples, the control unit 104E causes the display apparatus 14 to display a result of detection of the site region 116A and the lesion region 116B by the detection unit 104B in a display mode in accordance with the positional relationship (herein, the overlapping degree 124 as an example) between the site region 116A and the lesion region 116B. In this embodiment, as an example, the display mode in which the display apparatus 14 is caused to display the result of detection of the site region 116A and the lesion region 116B by the detection unit 104B is determined in accordance with the site indicated by the site region 116A, the type of the lesion (hereinafter also simply referred to as “lesion”), and the positional relationship between the site region 116A and the lesion region 116B (e.g., the consistency and the overlapping degree 124 between the site indicated in the site region 116A and the lesion).

More specifically, for example, the display mode in which the display apparatus 14 is caused to display the result of detection of the site region 116A by the detection unit 104B differs depending on the site indicated by the site region 116A, the lesion, and the positional relationship between the site region 116A and the lesion region 116B (e.g., the consistency and the overlapping degree 124 between the site indicated in the site region 116A and the lesion). In addition, for example, the display mode in which the display apparatus 14 is caused to display the result of detection of the lesion region 116B by the detection unit 104B is a mode in which the lesion region 116B is displayed on the first screen 22.

As illustrated in FIG. 12 as an example, the positional relationship identification unit 104D determines whether the overlapping degree 124 is greater than or equal to a preset overlapping degree (herein, “0.5” as an example). The preset overlapping degree may be a fixed value or may be a variable value that is changed in accordance with an instruction received by the reception apparatus 62 and/or various conditions. The preset overlapping degree is an example of a “first degree” according to the technology of the present disclosure.

The control unit 104E acquires the endoscopic image 114 and the ultrasound image 116 that is the determination target of the determination unit 104C, displays the acquired ultrasound image 116 on the first screen 22, and displays the acquired endoscopic image 114 on the second screen 24. In this case, if the positional relationship identification unit 104D determines that the overlapping degree 124 is less than the preset overlapping degree, the control unit 104E displays the ultrasound image 116 on the first screen 22 in the first display mode.

As illustrated in FIG. 13 as an example, the control unit 104E acquires the endoscopic image 114 and the ultrasound image 116 that is the determination target of the determination unit 104C, displays the acquired ultrasound image 116 on the first screen 22, and displays the acquired endoscopic image 114 on the second screen 24. In this case, if the positional relationship identification unit 104D determines that the overlapping degree 124 is greater than or equal to the preset overlapping degree, the control unit 104E displays the ultrasound image 116 on the first screen 22 in a second display mode. The second display mode is a mode in which the lesion region 116B is displayed so as to be identifiable in the ultrasound image 116, and the site region 116A is displayed so as to be comparable with the lesion region 116B. In the example illustrated in FIG. 13, as an example of the second display mode, a mode is illustrated in which the contour of the site region 116A and the contour of the lesion region 116B are bordered by curves, and the contour of the lesion region 116B is bordered by a thicker line than the contour of the site region 116A. In other words, the position of the site region 116A and the position of the lesion region 116B in the ultrasound image 116 are displayed so as to be identifiable, and the lesion region 116B is displayed in a state in which the site region 116A and the lesion region 116B are distinguishable by being displayed in a more emphasized manner than the site region 116A. The lesion region 116B being displayed in a more emphasized manner than the site region 116A means that the lesion region 116B is displayed in a more noticeable manner than the site region 116A.

In order to cause the display apparatus 14 to display the ultrasound image 116 in the second display mode (as an example, the display mode illustrated in FIG. 13), the control unit 104E acquires the coordinate information 118A from the site region information 118 and acquires the coordinate information 120A from the lesion region information 120. In addition, the control unit 104E processes the ultrasound image 116 on the basis of the coordinate information 118A and 120A, and displays it on the first screen 22. The processing performed on the ultrasound image 116 by the control unit 104E is processing for identifying the contour of the site region 116A from the coordinate information 118A and bordering the contour with a curve, identifying the contour of the lesion region 116B from the coordinate information 120A and bordering the contour with a curve, and making the contour of the lesion region 116B thicker than the contour of the site region 116A.

Although an example in which the contour of the lesion region 116B is made thicker than the contour of the site region 116A has been described herein, this is merely an example. For example, the luminance of the contour of the lesion region 116B may be made higher than the luminance of the contour of the site region 116A. In addition, for example, the lesion region 116B may be patterned or colored, and the site region 116A may be patterned or colored to be less noticeable than the lesion region 116B. In addition, for example, only the lesion region 116B out of the site region 116A and the lesion region 116B may be patterned or colored, and the contour of the site region 116A may be bordered by a curve. The site region 116A may be made more noticeable than the lesion region 116B by changing the line type of a curve bordering the contour of the site region 116A and the contour of the lesion region 116B. In this manner, any display mode may be employed as long as the site region 116A and the lesion region 116B are displayed so as to be identifiable and comparable (i.e., distinguishable).

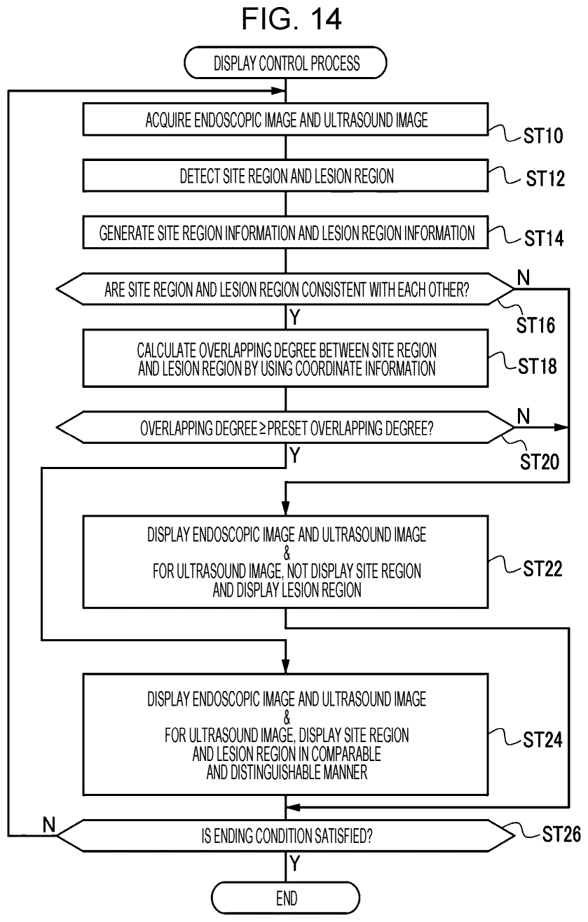

Next, an example of a flow of the display control process performed by the processor 104 of the display control apparatus 60 in a case where the reception apparatus 62 receives an instruction to start the execution of the display control process will be described with reference to FIG. 14. Note that the process flow illustrated in the flowchart in FIG. 14 is an example of an “image processing method” according to the technology of the present disclosure.

In the display control process illustrated in FIG. 14, first, in step ST10, the acquisition unit 104A acquires an endoscopic image 114 from the endoscope processing apparatus 54 and acquires an ultrasound image 116 from the ultrasound processing apparatus 58 (see FIG. 8). The endoscopic image 114 acquired by the acquisition unit 104A in step ST10 is an endoscopic image 114 of one frame that is yet to be used in step ST22 or step ST24, among the plurality of endoscopic images 114 included in the endoscopic moving image 28. In addition, the ultrasound image 116 acquired by the acquisition unit 104A in step ST10 is an ultrasound image 116 of one frame that is yet to be used in step ST12 and subsequent steps, among the plurality of ultrasound images 116 included in the ultrasound moving image 26. After step ST10 is executed, the display control process proceeds to step ST12.

In step ST12, the detection unit 104B performs AI image recognition processing on the ultrasound image 116 acquired in step ST10 to detect the site region 116A and the lesion region 116B from the ultrasound image 116 (see FIG. 9). After step ST12 is executed, the display control process proceeds to step ST14.

In step ST14, the detection unit 104B generates the site region information 118, which is information related to the site region 116A, and the lesion region information 120, which is information related to the lesion region 116B (see FIG. 9). After step ST14 is executed, the display control process proceeds to step ST16.

In step ST16, the determination unit 104C refers to the consistency determination table 122 and determines whether the site region 116A and the lesion region 116B are consistent with each other on the basis of the site region information 118 and the lesion region information 120 generated in step ST14 (see FIG. 9). If the site region 116A and the lesion region 116B are not consistent with each other in step ST16, the determination is negative, and the display control process proceeds to step ST22. If the site region 116A and the lesion region 116B are consistent with each other in step ST16, the determination is positive, and the display control process proceeds to step ST18.

In step ST18, the positional relationship identification unit 104D acquires the coordinate information 118A from the site region information 118 generated in step ST14, and acquires the coordinate information 120A from the lesion region information 120 generated in step ST14 (see FIG. 11). In addition, the positional relationship identification unit 104D calculates the overlapping degree 124 by using the coordinate information 118A and 120A (see FIG. 11). After step ST18 is executed, the display control process proceeds to step ST20.

In step ST20, the positional relationship identification unit 104D determines whether the overlapping degree 124 calculated in step ST18 is greater than or equal to the preset overlapping degree (see FIG. 12 and FIG. 13).

If the overlapping degree 124 is less than the preset overlapping degree in step ST20, the determination is negative, and the display control process proceeds to step ST22. If the overlapping degree 124 is greater than or equal to the preset overlapping degree in step ST20, the determination is positive, and the display control process proceeds to step ST24.

Note that the certainty of the lesion region 116B is determined by executing step ST16 to step ST20. If the determination in step ST16 is positive (i.e., the site region 116A and the lesion region 116B are consistent with each other) and the determination in step ST20 is positive (i.e., the site region 116A and the lesion region 116B have a known positional relationship), it is determined that the lesion region 116B is certain.

In step ST22, the control unit 104E displays the ultrasound image 116 acquired in step ST10 on the first screen 22 and displays the endoscopic image 114 acquired in step ST10 on the second screen 24. In this case, the control unit 104E displays the ultrasound image 116 in the first display mode. That is, the control unit 104E does not display the site region 116A and displays the lesion region 116B in the ultrasound image 116 (see FIG. 10 and FIG. 12). After step ST22 is executed, the display control process proceeds to step ST26.

In step ST24, the control unit 104E displays the ultrasound image 116 acquired in step ST10 on the first screen 22 and displays the endoscopic image 114 acquired in step ST10 on the second screen 24. In this case, the control unit 104E displays the ultrasound image 116 in the second display mode. That is, the control unit 104E displays the site region 116A and the lesion region 116B in the ultrasound image 116 in a comparable and distinguishable manner (see FIG. 13). After step ST24 is executed, the display control process proceeds to step ST26.

In step ST26, the control unit 104E determines whether a condition for ending the display control process (hereinafter referred to as a “display control process ending condition”) is satisfied. An example of the display control process ending condition is a condition that the reception apparatus 62 receives an instruction to end the display control process. If the display control process ending condition is not satisfied in step ST26, the determination is negative, and the display control process proceeds to step ST10. If the display control process ending condition is satisfied in step ST26, the determination is positive, and the display control process ends.

As described above, in the ultrasonic endoscope system 10, the detection unit 104B detects the site region 116A and the lesion region 116B from the ultrasound image 116. In this case, for example, if the site region 116A does not overlap with the lesion region 116B, there is a possibility that the lesion indicated by the lesion region 116B is a lesion irrelevant to the site indicated by the site region 116A. In addition, if the site region 116A and the lesion region 116B do not overlap with each other at all, there is a high possibility that the lesion indicated by the lesion region 116B is a lesion irrelevant to the site indicated by the site region 116A. In contrast, if the entirety of the lesion region 116B overlaps with the site region 116A, it can be said that the lesion indicated by the lesion region 116B is a lesion that is highly relevant to the site indicated by the site region 116A.

Thus, in the ultrasonic endoscope system 10, the certainty of the lesion region 116B is determined in accordance with the positional relationship between the site region 116A and the lesion region 116B (see step ST20 in FIG. 14). Then, the determination result is displayed on the first screen 22 (see FIG. 12 and FIG. 13). That is, the result of detection of the site region 116A and the lesion region 116B from the ultrasound image 116 by the detection unit 104B is displayed on the first screen 22 in a display mode in accordance with the positional relationship between the site region 116A and the lesion region 116B (see FIG. 12 and FIG. 13). Accordingly, a user or the like can grasp the lesion with high accuracy. For example, the user can grasp the lesion with high accuracy as compared with a case where the result of detection of the site region 116A and the lesion region 116B is always displayed in a fixed display mode regardless of the positional relationship between the site region 116A and the lesion region 116B.

In addition, in the ultrasonic endoscope system 10, the display mode for displaying, on the first screen 22, the result of detection of the site region 116A and the lesion region 116B from the ultrasound image 116 by the detection unit 104B is determined in accordance with the site, the lesion, and the positional relationship between the site region 116A and the lesion region 116B. Thus, the display apparatus 14 displays the result of detection of the site region 116A and the lesion region 116B from the ultrasound image 116 by the detection unit 104B in a display mode in accordance with the site, the lesion, and the positional relationship between the site region 116A and the lesion region 116B (see FIG. 10, FIG. 12, and FIG. 13). Accordingly, a user or the like can grasp the lesion with high accuracy. For example, the user can grasp the lesion with high accuracy as compared with a case where the result of detection of the site region 116A and the lesion region 116B are always displayed in a fixed display mode regardless of the site, the lesion, and the positional relationship between the site region 116A and the lesion region 116B.

In addition, in the ultrasonic endoscope system 10, the display mode in which the result of detection of the site region 116A and the lesion region 116B from the ultrasound image 116 by the detection unit 104B is displayed on the first screen 22 is determined in accordance with the positional relationship between the site region 116A and the lesion region 116B and the consistency between the site and the lesion. Thus, the display apparatus 14 displays the result of detection of the site region 116A and the lesion region 116B from the ultrasound image 116 by the detection unit 104B in a display mode in accordance with the positional relationship between the site region 116A and the lesion region 116B and the consistency between the site and the lesion (see FIG. 10, FIG. 12, and FIG. 13). Accordingly, a user or the like can grasp the lesion with high accuracy. For example, the user can grasp the lesion with high accuracy as compared with a case where the result of detection of the site region 116A and the lesion region 116B are always displayed in a fixed display mode regardless of the positional relationship between the site region 116A and the lesion region 116B and the consistency between the site and the lesion.

In addition, in the ultrasonic endoscope system 10, the display mode of the site region 116A differs depending on the site, the lesion, and the positional relationship between the site region 116A and the lesion region 116B, and the lesion region 116B is displayed on the first screen 22 (see FIG. 10, FIG. 12, and FIG. 13). For example, in the examples illustrated in FIG. 10 and FIG. 12, the lesion region 116B is displayed on the first screen 22, but the site region 116A is not displayed on the first screen 22, whereas in the example illustrated in FIG. 13, the site region 116A and the lesion region 116B are displayed on the first screen 22. Thus, a user or the like can easily recognize a difference between the site and the lesion. For example, the user or the like can easily recognize the difference between the site and the lesion as compared with a case where the display mode of the site region 116A is always fixed regardless of the site, the lesion, and the positional relationship between the site region 116A and the lesion region 116B.

In addition, in the ultrasonic endoscope system 10, if the site and the lesion are not consistent with each other, the site region 116A is not displayed on the first screen 22, and the lesion region 116B is displayed on the first screen 22 (see FIG. 10). This can suppress erroneous recognition of a site as a lesion or a lesion as a site. For example, it is possible to suppress erroneous recognition of a site as a lesion or a lesion as a site as compared with a case where both the site region 116A and the lesion region 116B are displayed although the site and the lesion are not consistent with each other.

In addition, in the ultrasonic endoscope system 10, the positional relationship between the site region 116A and the lesion region 116B is defined by the overlapping degree 124. Thus, it is possible to display, on the first screen 22, the result of detection of the site region 116A and the lesion region 116B from the ultrasound image 116 by the detection unit 104B in a display mode corresponding to the overlapping degree 124 (see FIG. 12 and FIG. 13).

In addition, in the ultrasonic endoscope system 10, the positional relationship between the site region 116A and the lesion region 116B is defined by the overlapping degree 124, and if the overlapping degree 124 is greater than or equal to the preset overlapping degree, the lesion region 116B is displayed so as to be identifiable in the ultrasound image 116 (see FIG. 13). Accordingly, a user or the like can grasp a lesion that is highly relevant to the site captured in the ultrasound image 116.

In addition, in the ultrasonic endoscope system 10, the positional relationship between the site region 116A and the lesion region 116B is defined by the overlapping degree 124, and if the overlapping degree 124 is greater than or equal to the preset overlapping degree, the lesion region 116B is displayed so as to be identifiable in the ultrasound image 116, and the site region 116A is displayed so as to be comparable with the lesion region 116B. Accordingly, a user or the like can grasp the positional relationship between the site and the lesion that is highly relevant to the site.

In addition, in the ultrasonic endoscope system 10, the ultrasound moving image 26 is a moving image defined by the plurality of ultrasound images 116, the detection unit 104B detects the site region 116A and the lesion region 116B for each ultrasound image 116, and the display mode of the site region 116A and the lesion region 116B is determined for each ultrasound image 116. Accordingly, even if the ultrasound moving image 26 is a moving image defined by the plurality of ultrasound images 116, a user or the like can grasp the lesion site for each ultrasound image 116.

In addition, in the ultrasonic endoscope system 10, through step ST16 to step ST20, the certainty of the lesion region 116B is determined. For example, if the site region 116A and the lesion region 116B are consistent with each other and the site region 116A and the lesion region 116B have a known positional relationship (e.g., step ST20: Y), the lesion region 116B is determined to be certain. In addition, the site region 116A and the lesion region 116B in the ultrasound image 116 are displayed in a comparable and distinguishable manner (see FIG. 13). Accordingly, a user or the like can grasp the highly relevant site and lesion.

### First Modification

Although the above embodiment has described examples of forms in which the site region 116A is displayed or is not displayed if the combination of the site region 116A and the lesion region 116B is consistent (see FIG. 11 to FIG. 13), the technology of the present disclosure is not limited to this. For example, if the combination of the site region 116A and the lesion region 116B is consistent, the display mode of the site region 116A may be a mode in which the site region 116A is displayed on the first screen 22 and which is determined in accordance with the positional relationship between the site region 116A and the lesion region 116B.

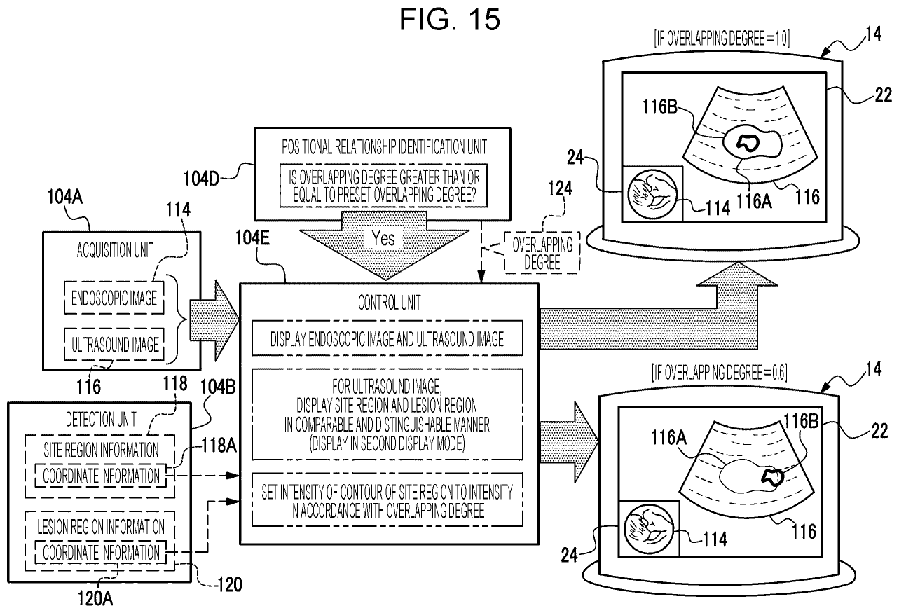

In this case, as illustrated in FIG. 15 as an example, if the positional relationship identification unit 104D determines that the overlapping degree 124 is greater than or equal to the preset overlapping degree, the control unit 104E displays the ultrasound image 116 on the first screen 22 in the second display mode and sets the intensity of the contour of the site region 116A to an intensity in accordance with the overlapping degree 124. For example, as the overlapping degree 124 is larger, the contour is made more noticeable.

Examples of a method for making the contour noticeable include a method for increasing the luminance of the contour and a method for increasing the thickness of the contour. In the example illustrated in FIG. 15, since the contour of the site region 116A displayed on the first screen 22 when the overlapping degree is “1.0” is thicker than the contour of the site region 116A displayed on the first screen 22 when the overlapping degree is “0.6”, the site region 116A displayed on the first screen 22 when the overlapping degree is “1.0” is more noticeable than the site region 116A displayed on the first screen 22 when the overlapping degree is “0.6”. Accordingly, a user or the like can recognize the positional relationship (e.g., the overlapping degree 124) between the site and the lesion that are consistent with each other.

### Second Modification

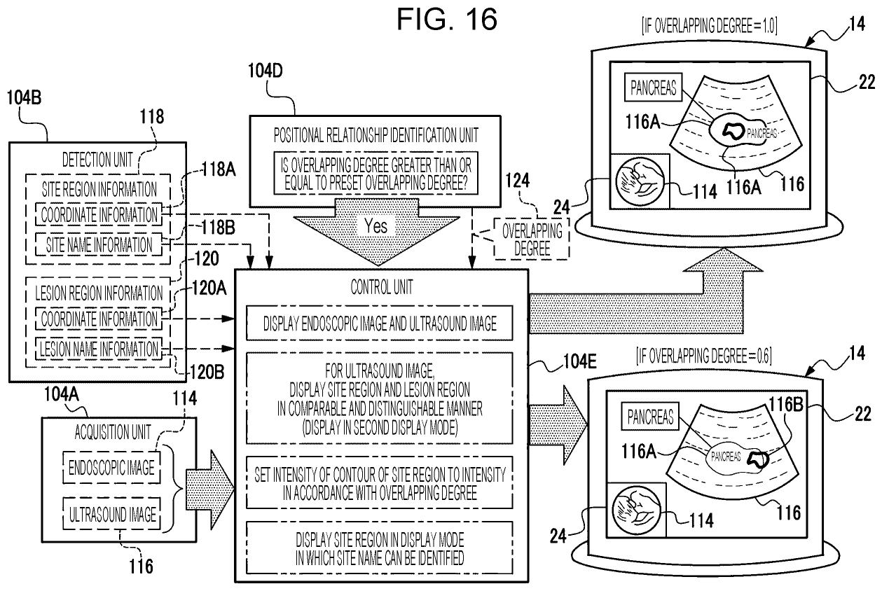

Although the name of the site indicated by the site region 116A is not displayed on the first screen 22 in the above embodiment, the technology of the present disclosure is not limited to this, and, for example, as illustrated in FIG. 16, information indicating the name of the site indicated by the site region 116A may be displayed on the first screen 22.

In this case, the control unit 104E acquires the site name information 118B from the site region information 118, and displays the information indicating the name of the site identified from the site name information 118B so as to be superimposed on the site region 116A on the first screen 22. In the example illustrated in FIG. 16, characters “pancreas” are displayed so as to be superimposed on the site region 116A as the information indicating the name of the site. Instead of characters, numbers, marks, figures, or the like by which the name of the site (i.e., the type of the site) can be identified may be used. In addition, not limited to the superimposed display, as illustrated in FIG. 16, the information indicating the name of the site (characters “pancreas” in the example illustrated in FIG. 16) may be displayed in a pop-up manner from the site region 116A. In addition, in accordance with an instruction received by the reception apparatus 62, the control unit 104E may switch between display and non-display of the information indicating the name of the site. In this manner, by displaying the information indicating the name of the site in association with the site region 116A, a user or the like can grasp the name of the site indicated by the site region 116A displayed on the first screen 22.

In addition, the control unit 104E may acquire the lesion name information 120B from the lesion region information 120 and may display information indicating the name of the lesion identified from the lesion name information 120B so as to be superimposed on the lesion region 116B on the first screen 22. In addition, without limitation to the superimposed display, the information indicating the name of the lesion may be displayed in a pop-up manner from the lesion region 116B. In addition, in accordance with an instruction received by the reception apparatus 62, the control unit 104E may switch between display and non-display of the information indicating the name of the lesion. In this manner, by displaying the information indicating the name of the lesion in association with the lesion region 116B, a user or the like can grasp the name of the lesion indicated by the lesion region 116B displayed on the first screen 22.

### Third Modification

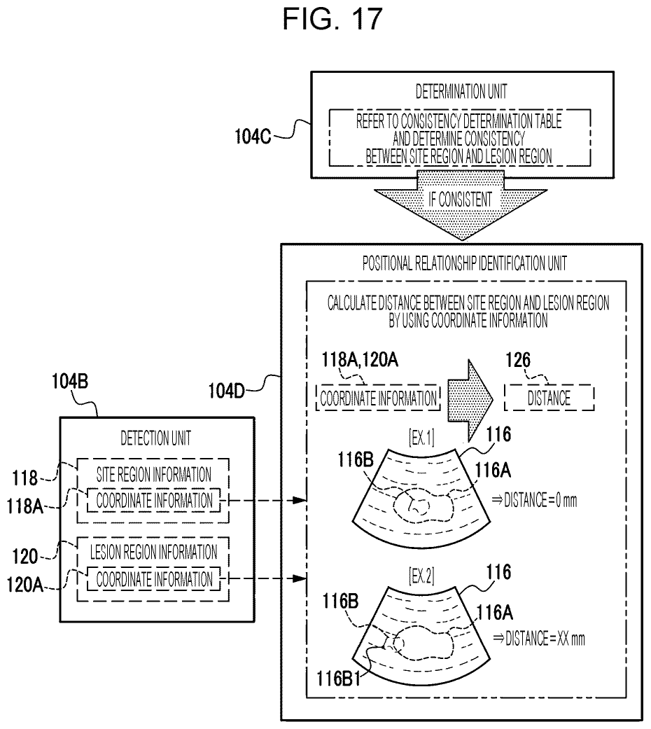

Although the above embodiment has described the overlapping degree 124 as an example, this is merely an example, and for example, as illustrated in FIG. 17, the positional relationship identification unit 104D may calculate a distance 126 instead of the overlapping degree 124. As in the case of the overlapping degree 124, the distance 126 is calculated by using the coordinate information 118A and 120A. If the overlapping degree 124 is “1.0”, that is, if the entirety of the lesion region 116B overlaps with the site region 116A, the distance 126 is zero millimeters. If there is a non-overlapping region between the site region 116A and the lesion region 116B, the distance 126 exceeds zero millimeters. An example of the distance 126 is the distance between the site region 116A and part of the contour of a region of the lesion region 116B not overlapping with the site region 116A. For example, the part of the contour of the region not overlapping with the site region 116A is a position 116B1 farthest from the site region 116A in the contour of the region not overlapping with the site region 116A. In this manner, even if the distance 126 is used instead of the overlapping degree 124, substantially the same effects as those in the above embodiment are obtained.

### Fourth Modification

Although the above embodiment has described an example of a form in which the display mode of the ultrasound image 116 is determined in accordance with the site, the lesion, and the positional relationship between the site region 116A and the lesion region 116B, the technology of the present disclosure is not limited to this. For example, the display mode of the ultrasound image 116 may be determined in accordance with a certainty factor for a result of detection of the site region 116A by the AI image recognition processing, a certainty factor for a result of detection of the lesion region 116B by the AI image recognition processing, and the positional relationship between the site region 116A and the lesion region 116B.

Herein, an example of the certainty factor for the result of detection of the site region 116A by the AI image recognition processing is a value corresponding to a maximum score among a plurality of scores obtained from a trained model obtained by causing a neural network to perform machine learning for detecting the site region 116A. In addition, an example of the certainty factor for the result of detection of the lesion region 116B by the AI image recognition processing is a value corresponding to a maximum score among a plurality of scores obtained from a trained model obtained by causing a neural network to perform machine learning for detecting the lesion region 116B. An example of the value corresponding to the score is a value (i.e., a probability expressed by a value in a range of 0 to 1) obtained by converting the score by an activation function used as an output layer of the neural network. An example of the activation function is a softmax function used as an output layer of multi-class classification.

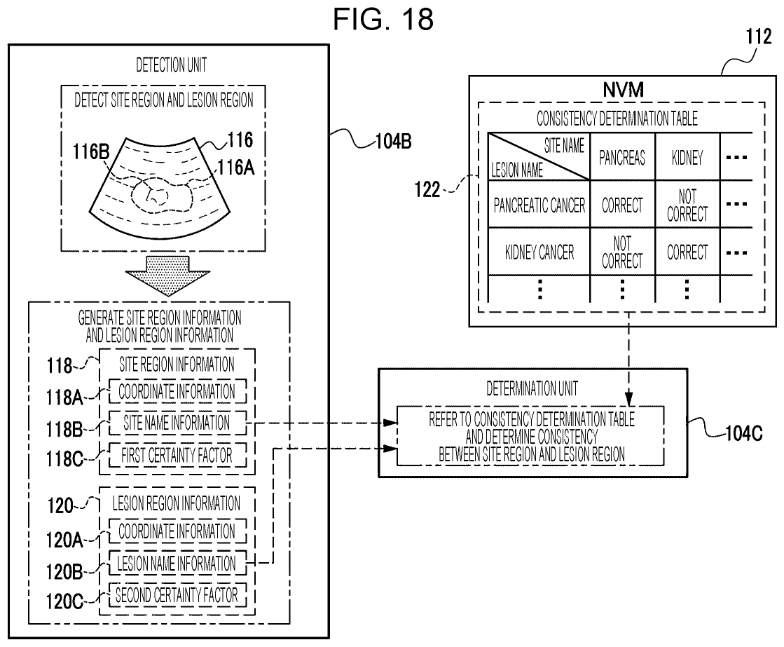

As illustrated in FIG. 18 as an example, the detection unit 104B acquires a first certainty factor 118C and a second certainty factor 120C used in the AI image recognition processing on the ultrasound image 116. The first certainty factor 118C is a certainty factor for the result of detection of the site region 116A by the AI image recognition processing. The second certainty factor 120C is a certainty factor for the result of detection of the lesion region 116B by the AI image recognition processing. The detection unit 104B generates information including the coordinate information 118A, the site name information 118B, and the first certainty factor 118C as the site region information 118. In addition, the detection unit 104B generates information including the coordinate information 120A, the lesion name information 120B, and the second certainty factor 120C as the lesion region information 120. The determination unit 104C determines the consistency between the site region 116A and the lesion region 116B in the same manner as in the above embodiment.

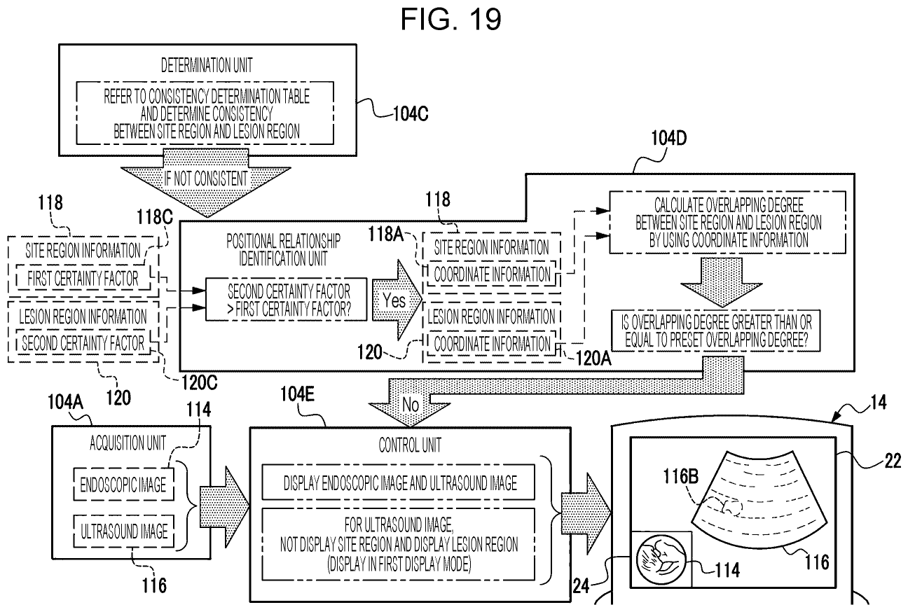

As illustrated in FIG. 19 to FIG. 24 as examples, the display mode of the ultrasound image 116 is determined in accordance with a magnitude relationship between the first certainty factor 118C and the second certainty factor 120C and the positional relationship between the site region 116A and the lesion region 116B.

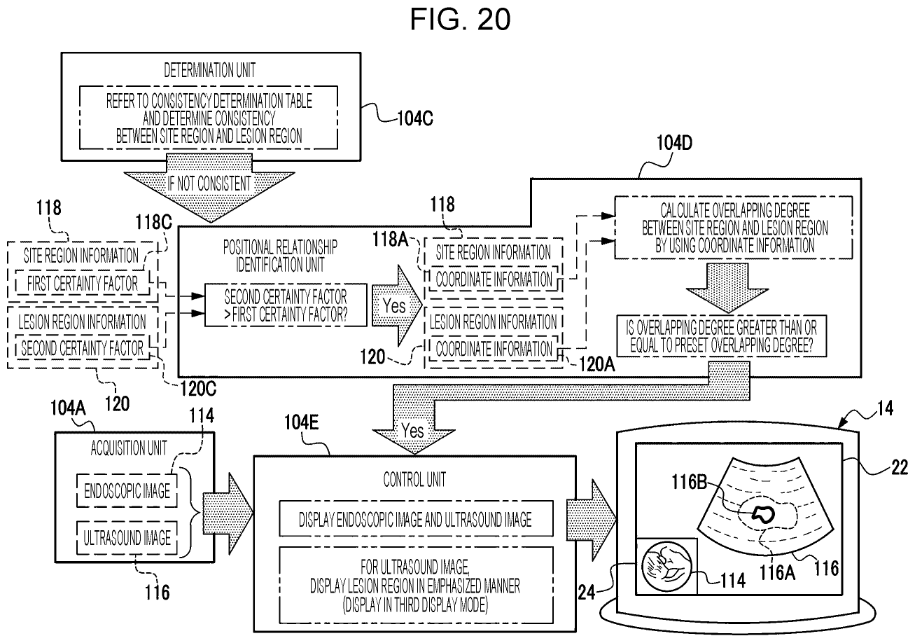

As illustrated in FIG. 19 and FIG. 20 as examples, if the determination unit 104C determines that the site region 116A and the lesion region 116B are not consistent with each other, the positional relationship identification unit 104D determines whether the second certainty factor 120C included in the lesion region information 120 is greater than the first certainty factor 118C included in the site region information 118. If the second certainty factor 120C is greater than the first certainty factor 118C, the positional relationship identification unit 104D calculates the overlapping degree 124 in the same manner as in the above embodiment and determines whether the overlapping degree 124 is greater than or equal to the preset overlapping degree.

In this case, as illustrated in FIG. 19 as an example, if the positional relationship identification unit 104D determines that the overlapping degree 124 is less than the preset overlapping degree, the control unit 104E displays the endoscopic image 114 on the second screen 24 and displays the ultrasound image 116 on the first screen 22 in the first display mode. In contrast, as illustrated in FIG. 20 as an example, if the positional relationship identification unit 104D determines that the overlapping degree 124 is greater than or equal to the preset overlapping degree, the control unit 104E displays the endoscopic image 114 on the second screen 24 and displays the ultrasound image 116 on the first screen 22 in a third display mode. The third display mode is a mode in which the lesion region 116B is displayed in an emphasized manner. Examples of a method for displaying the lesion region 116B in an emphasized manner include a method for increasing the luminance of the contour of the lesion region 116B, a method for coloring or patterning the lesion region 116B, a method for hiding a region other than the lesion region 116B in the ultrasound image 116, and the like. In this manner, the lesion region 116B is displayed in an emphasized manner by being displayed in a mode to be distinguishable from other regions in the ultrasound image 116.

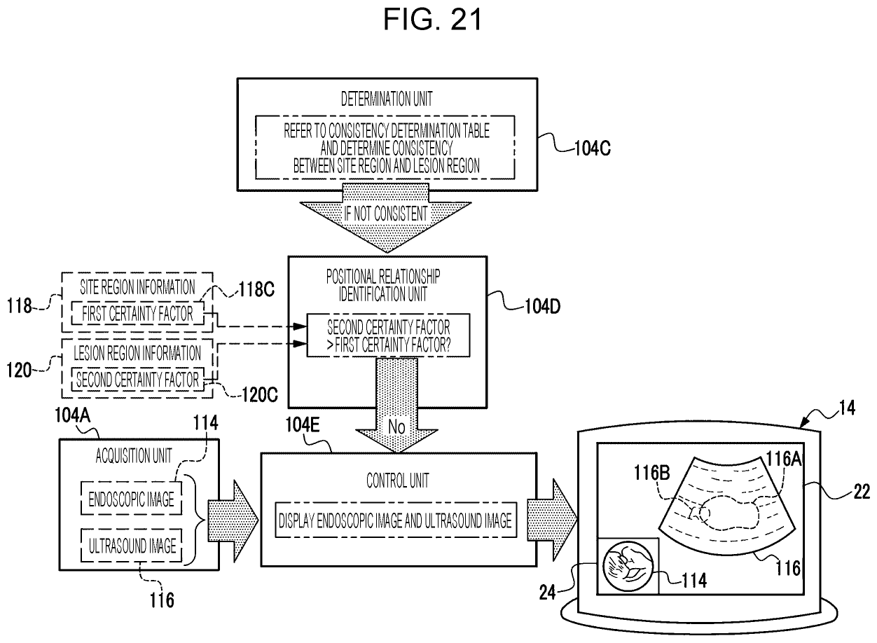

As illustrated in FIG. 21 as an example, if the determination unit 104C determines that the site region 116A and the lesion region 116B are not consistent with each other, the positional relationship identification unit 104D determines whether the second certainty factor 120C included in the lesion region information 120 is greater than the first certainty factor 118C included in the site region information 118. In this case, if the positional relationship identification unit 104D determines that the second certainty factor 120C is less than or equal to the first certainty factor, the control unit 104E displays the endoscopic image 114 acquired by the acquisition unit 104A on the second screen 24 and displays the ultrasound image 116 acquired by the acquisition unit 104A on the first screen 22.

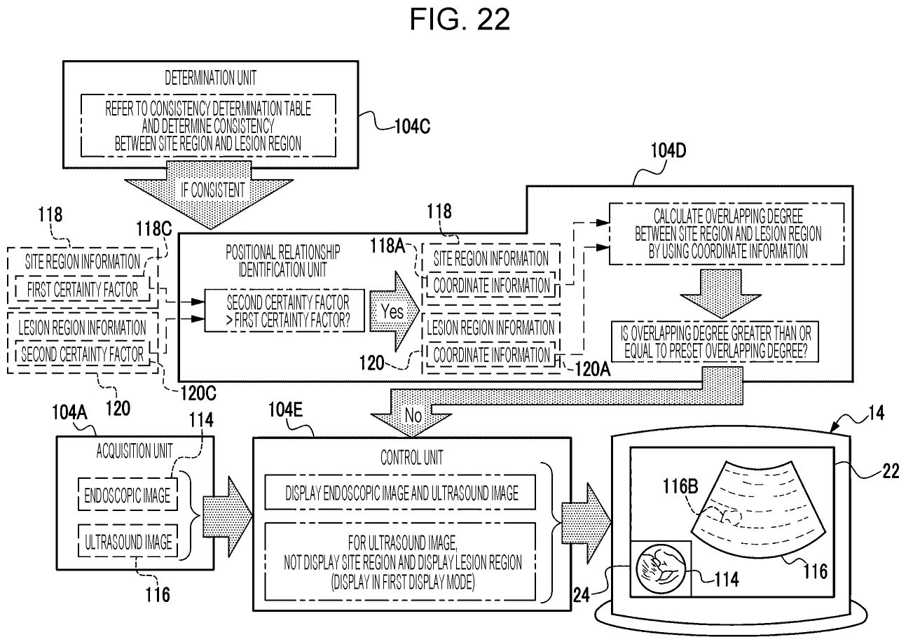

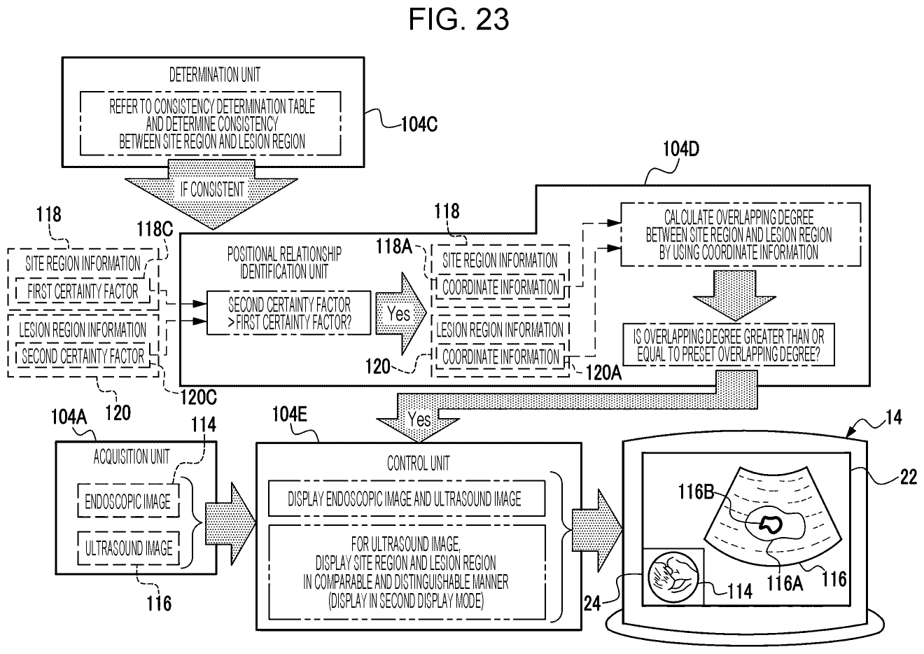

As illustrated in FIG. 22 and FIG. 23 as examples, if the determination unit 104C determines that the site region 116A and the lesion region 116B are consistent with each other, the positional relationship identification unit 104D determines whether the second certainty factor 120C included in the lesion region information 120 is greater than the first certainty factor 118C included in the site region information 118. If the second certainty factor 120C is greater than the first certainty factor, the positional relationship identification unit 104D calculates the overlapping degree 124 in the same manner as in the above embodiment, and determines whether the overlapping degree 124 is greater than or equal to the preset overlapping degree.

In this case, as illustrated in FIG. 22 as an example, if the positional relationship identification unit 104D determines that the overlapping degree 124 is less than the preset overlapping degree, the control unit 104E displays the endoscopic image 114 on the second screen 24 and displays the ultrasound image 116 on the first screen 22 in the first display mode. In contrast, as illustrated in FIG. 23 as an example, if the positional relationship identification unit 104D determines that the overlapping degree 124 is greater than or equal to the preset overlapping degree, the control unit 104E displays the ultrasound image 116 on the first screen 22 in the second display mode.

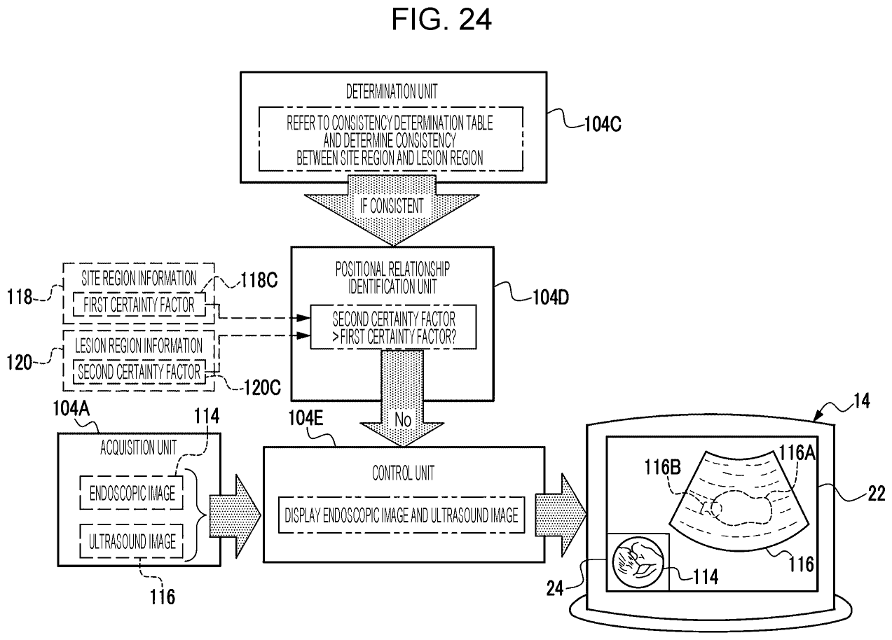

As illustrated in FIG. 24 as an example, if the determination unit 104C determines that the site region 116A and the lesion region 116B are consistent with each other, the positional relationship identification unit 104D determines whether the second certainty factor 120C included in the lesion region information 120 is greater than the first certainty factor 118C included in the site region information 118. In this case, if the detection unit 104B determines that the second certainty factor 120C is less than or equal to the first certainty factor, the control unit 104E displays the endoscopic image 114 acquired by the acquisition unit 104A on the second screen 24 and displays the ultrasound image 116 acquired by the acquisition unit 104A on the first screen 22.

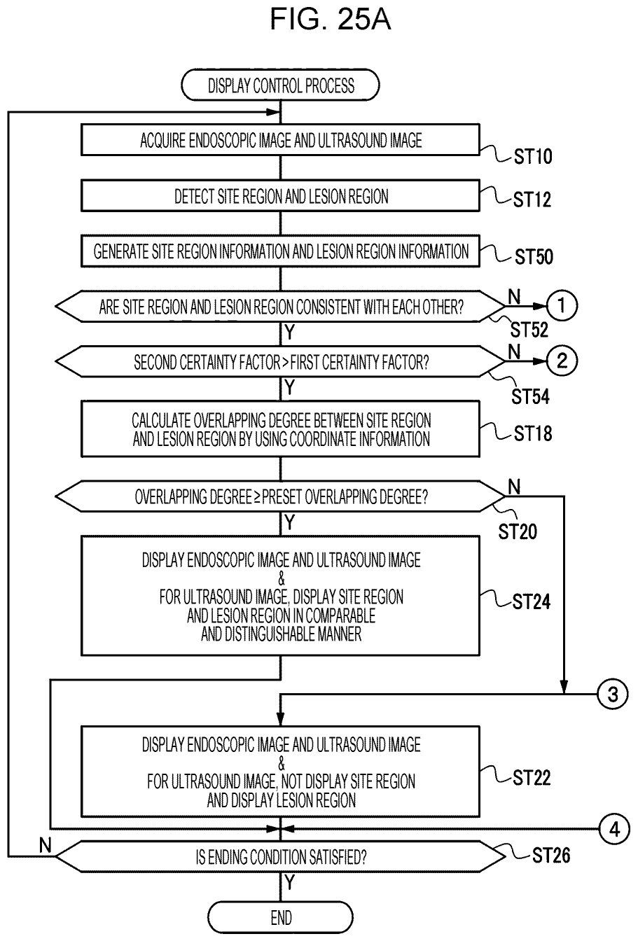

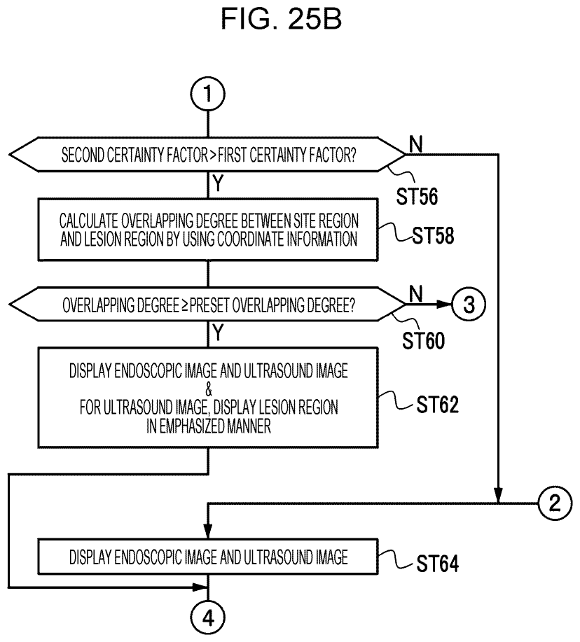

Next, an example of a flow of a display control process performed by the processor 104 of the display control apparatus 60 in a case where the reception apparatus 62 receives an instruction to start the execution of the display control process according to the fourth modification will be described with reference to FIG. 25A and FIG. 25B.

The flowcharts illustrated in FIG. 25A and FIG. 25B are different from the flowchart illustrated in FIG. 14 in that step ST50 to step ST64 are applied instead of step ST14 and step ST16. Note that the same steps as those in the flowchart illustrated in FIG. 14 are denoted by the same step numbers, and description thereof is omitted herein.

In the display control process illustrated in FIG. 25A, in step ST50, the detection unit 104B generates information including the coordinate information 118A, the site name information 118B, and the first certainty factor 118C as the site region information 118. In addition, the detection unit 104B generates information including the coordinate information 120A, the lesion name information 120B, and the second certainty factor 120C as the lesion region information 120. After step ST50 is executed, the display control process proceeds to step ST52.

In step ST52, the determination unit 104C refers to the consistency determination table 122 and determines whether the site region 116A and the lesion region 116B are consistent with each other on the basis of the site region information 118 and the lesion region information 120 generated in step ST50 (see FIG. 18). If the site region 116A and the lesion region 116B are not consistent with each other in step ST52, the determination is negative, and the display control process proceeds to step ST56 illustrated in FIG. 25B. If the site region 116A and the lesion region 116B are consistent with each other in step ST52, the determination is positive, and the display control process proceeds to step ST54.

In step ST54, the positional relationship identification unit 104D acquires the first certainty factor 118C from the site region information 118 generated in step ST50, and acquires the second certainty factor 120C from the lesion region information 120 generated in step ST50. In addition, the positional relationship identification unit 104D determines whether the second certainty factor 120C is greater than the first certainty factor 118C. If the second certainty factor 120C is less than or equal to the first certainty factor 118C in step ST54, the determination is negative, and the process proceeds to step ST64 illustrated in FIG. 25B. If the second certainty factor 120C is greater than the first certainty factor 118C in step ST54, the determination is positive, and the display control process proceeds to step ST18.

In step ST56 illustrated in FIG. 25B, the positional relationship identification unit 104D acquires the first certainty factor 118C from the site region information 118 generated in step ST50, and acquires the second certainty factor 120C from the lesion region information 120 generated in step ST50. In addition, the positional relationship identification unit 104D determines whether the second certainty factor 120C is greater than the first certainty factor 118C. If the second certainty factor 120C is less than or equal to the first certainty factor 118C in step ST56, the determination is negative, and the process proceeds to step ST64. If the second certainty factor 120C is greater than the first certainty factor 118C in step ST56, the determination is positive, and the display control process proceeds to step ST58.

In step ST58, the positional relationship identification unit 104D acquires the coordinate information 118A from the site region information 118 generated in step ST50, and acquires the coordinate information 120A from the lesion region information 120 generated in step ST50 (see FIG. 19 and FIG. 20). In addition, the positional relationship identification unit 104D calculates the overlapping degree 124 by using the coordinate information 118A and 120A (see FIG. 19 and FIG. 20). After step ST58 is executed, the display control process proceeds to step ST60.

In step ST60, the positional relationship identification unit 104D determines whether the overlapping degree 124 calculated in step ST58 is greater than or equal to the preset overlapping degree. If the overlapping degree 124 is less than the preset overlapping degree in step ST60, the determination is negative, and the display control process proceeds to step ST22 illustrated in FIG. 25A. If the overlapping degree 124 is greater than or equal to the preset overlapping degree in step ST60, the determination is positive, and the display control process proceeds to step ST62.

In step ST62, the control unit 104E displays the ultrasound image 116 acquired in step ST10 on the first screen 22 and displays the endoscopic image 114 acquired in step ST10 on the second screen 24. In this case, the control unit 104E displays the ultrasound image 116 in the third display mode. That is, the control unit 104E displays the lesion region 116B in the ultrasound image 116 in an emphasized manner (see FIG. 20). After step ST62 is executed, the display control process proceeds to step ST26 illustrated in FIG. 25A.

In step ST64, the control unit 104E displays the ultrasound image 116 acquired in step ST10 on the first screen 22 and displays the endoscopic image 114 acquired in step ST10 on the second screen 24. After step ST64 is executed, the display control process proceeds to step ST26 illustrated in FIG. 25A.

As described above, in the fourth modification, the result of detection of the site region 116A and the lesion region 116B from the ultrasound image 116 by the detection unit 104B is displayed on the first screen 22 in a display mode in accordance with the first certainty factor 118C, the second certainty factor 120C, and the positional relationship (e.g., the overlapping degree 124) between the site region 116A and the lesion region 116B. Accordingly, it is possible to suppress the occurrence of a situation in which a user or the like recognizes a site and a lesion having low relevance to each other.

For example, it is possible to suppress the occurrence of a situation in which a user or the like recognizes a site and a lesion having low relevance to each other as compared with a case where the display mode of the ultrasound image 116 is determined without considering the first certainty factor 118C and the second certainty factor 120C at all.

In addition, in the fourth modification, the result of detection of the site region 116A and the lesion region 116B from the ultrasound image 116 by the detection unit 104B is displayed on the first screen 22 in a display mode in accordance with the magnitude relationship between the first certainty factor 118C and the second certainty factor 120C and the positional relationship (e.g., the overlapping degree 124) between the site region 116A and the lesion region 116B. Accordingly, it is possible to suppress the occurrence of a situation in which a user or the like recognizes a site and a lesion having low relevance to each other. For example, it is possible to suppress the occurrence of a situation in which a user or the like recognizes a site and a lesion having low relevance to each other as compared with a case where the display mode of the ultrasound image 116 is determined without considering the magnitude relationship between the first certainty factor 118C and the second certainty factor 120C and the positional relationship between the site region 116A and the lesion region 116B at all. In addition, a user or the like can perceive the magnitude relationship between the first certainty factor 118C and the second certainty factor 120C through the display mode of the first screen 22. In addition, since the target to be compared with the second certainty factor 120C is the first certainty factor 118C, it is not necessary to prepare a threshold value to be compared with the second certainty factor 120C in advance.

Note that, although the display mode is determined in accordance with the magnitude relationship between the first certainty factor 118C and the second certainty factor 120C in the fourth modification, the display mode is not limited to this and may be determined in accordance with whether the second certainty factor 120C is greater than or equal to a preset certainty factor (e.g., 0.7). The preset certainty factor may be a fixed value or may be a variable value that is changed in accordance with an instruction received by the reception apparatus 62 and/or various conditions. If the second certainty factor 120C is greater than or equal to the preset certainty factor, the lesion region 116B is displayed in an emphasized manner compared with a case where the second certainty factor 120C is less than the preset certainty factor. In addition, a display intensity of the lesion region 116B may be determined in accordance with the magnitude of the second certainty factor 120C. For example, as the second certainty factor 120C is larger, the display intensity of the lesion region 116B is increased.

In addition, in a case where the display intensity of the lesion region 116B is increased in accordance with the overlapping degree 124, whether the display intensity is determined in accordance with the magnitude of the second certainty factor 120C or the display intensity is determined in accordance with the overlapping degree 124 may be identified by a display mode (e.g., the color of the contour of the site region 116A and/or the contour of the lesion region 116B). Note that the display intensity of the site region 116A may also be determined by a similar method using the first certainty factor 118C.

### Fifth Modification

Although the above embodiment has described an example of a form in which the display mode of the ultrasound image 116 is determined in accordance with the positional relationship between the one site region 116A and the lesion region 116B, the technology of the present disclosure is not limited to this. For example, the display mode of the ultrasound image 116 may be determined in accordance with a plurality of positional relationships. The plurality of positional relationships herein are positional relationships between a plurality of site regions for a plurality of types of sites and the lesion region 116B.

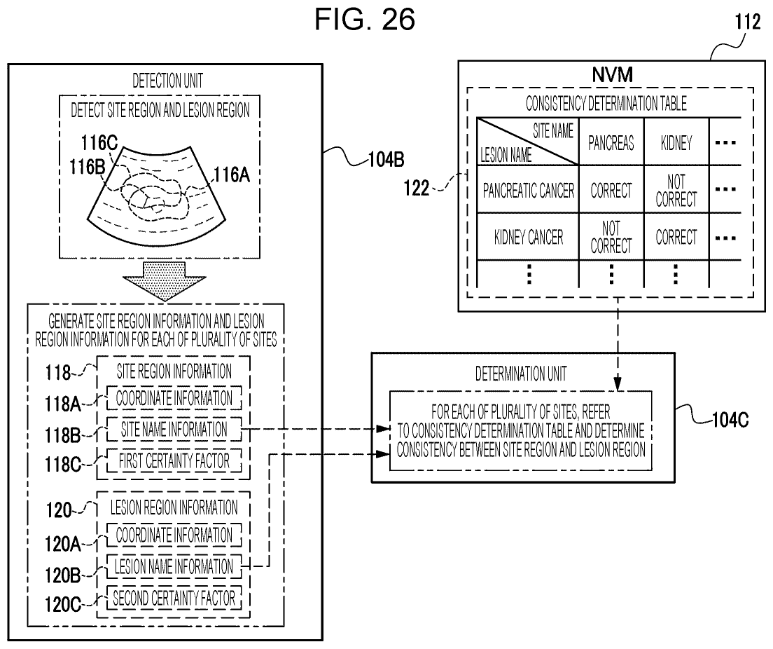

As illustrated in FIG. 26 as an example, in the same manner as in the above embodiment, the detection unit 104B detects the site region 116A, the lesion region 116B, and a site region 116C from the ultrasound image 116. The site indicated by the site region 116A and a site indicated by the site region 116C are different types of sites from each other. For example, the site indicated by the site region 116A is a pancreas, and the site indicated by the site region 116C is a duodenum.

The detection unit 104B generates the lesion region information 120 in the same manner as in the above embodiment. In addition, the detection unit 104B generates the site region information 118 for each of the plurality of sites. In the example illustrated in FIG. 26, the site region information 118 related to the site region 116A and site region information 118 related to the site region 116C are generated by the detection unit 104B.

The determination unit 104C refers to the consistency determination table 122 and determines the consistency between the site region 116A and the lesion region 116B and the consistency between the site region 116C and the lesion region 116B. In the ultrasonic endoscope system 10 according to the fifth modification, the display control process is performed based on the plurality of pieces of site region information 118 generated by the detection unit 104B, the lesion region information 120 generated by the detection unit 104B, and the determination result obtained by the determination unit 104C.

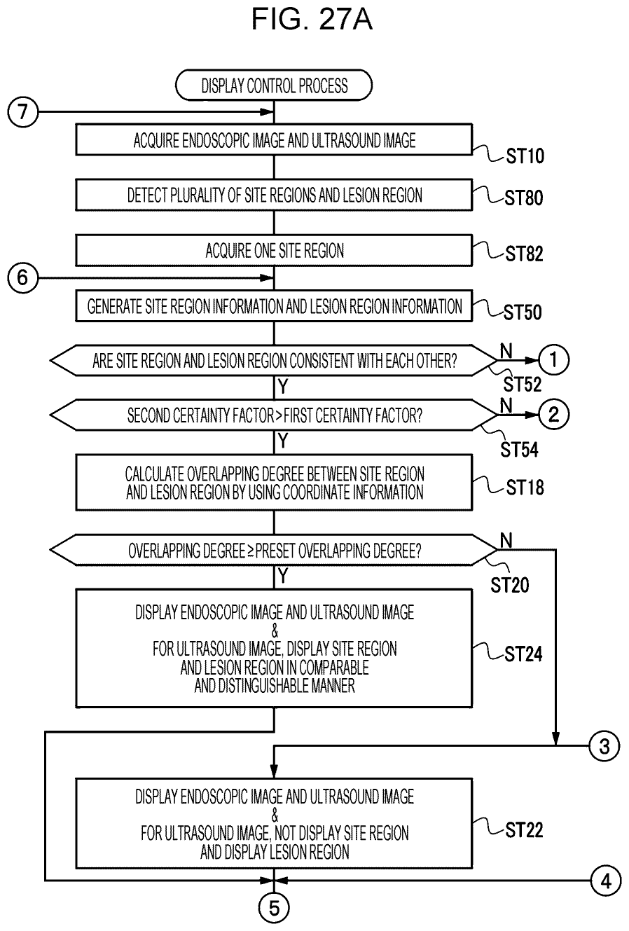

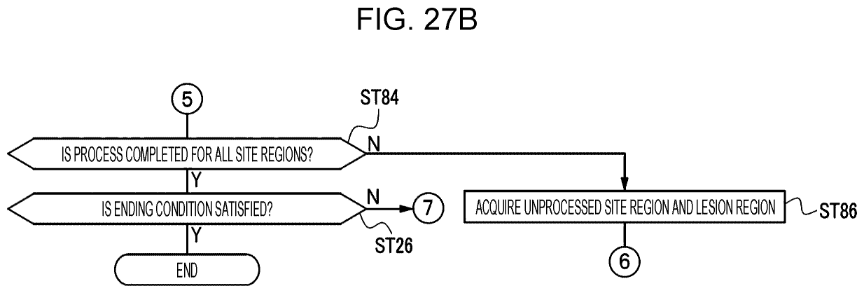

Next, an example of a flow of a display control process performed by the processor 104 of the display control apparatus 60 in a case where the reception apparatus 62 receives an instruction to start th execution of the display control process according to the fifth modification will be described with reference to FIG. 27A and FIG. 27B.

The flowcharts illustrated in FIG. 27A and FIG. 27B are different from the flowcharts illustrated in FIG. 25A and FIG. 25B in that step ST80 is applied instead of step ST12, step ST82 is inserted between step ST80 and step ST50, and step ST84 and step ST86 are inserted between step ST22 and step ST26. Note that the same steps as those in the flowcharts illustrated in FIG. 25A and FIG. 25B are denoted by the same step numbers, and description thereof is omitted herein.

In the display control process illustrated in FIG. 27A, in step ST80, the detection unit 104B performs AI image recognition processing to detect a plurality of site regions (herein, as an example, the site regions 116A and 116C) and the lesion region 116B from the ultrasound image 116. After step ST80 is executed, the display control process proceeds to step ST82.

In step ST82, the detection unit 104B acquires one site region that is not used in step ST50 and subsequent steps from the plurality of site regions detected in step ST80. After step ST82 is executed, the display control process proceeds to step ST50. In step ST50 and subsequent steps, the process is performed by using the one site region acquired in step ST82 or step ST86 illustrated in FIG. 27B.

In step ST84 illustrated in FIG. 27B, the control unit 104E determines whether step ST50 and subsequent steps have been executed for all the site regions detected in step ST80. In step ST84, if step ST50 and subsequent steps have not been executed for all the site regions detected in step ST80, the determination is negative, and the display control process proceeds to step ST86. In step ST84, if step ST50 and subsequent steps have been executed for all the site regions detected in step ST80, the determination is positive, and the display control process proceeds to step ST26.

In step ST86, the detection unit 104B acquires one site region that has not been used in step ST50 and subsequent steps from the plurality of site regions detected in step ST80. After step ST86 is executed, the display control process proceeds to step ST50 illustrated in FIG. 27A.

In this manner, by performing the display control process illustrated in FIG. 27A and FIG. 27B, the ultrasound image 116 is displayed in a display mode determined in accordance with, for example, the positional relationship between each of the plurality of site regions and the lesion region 116B.

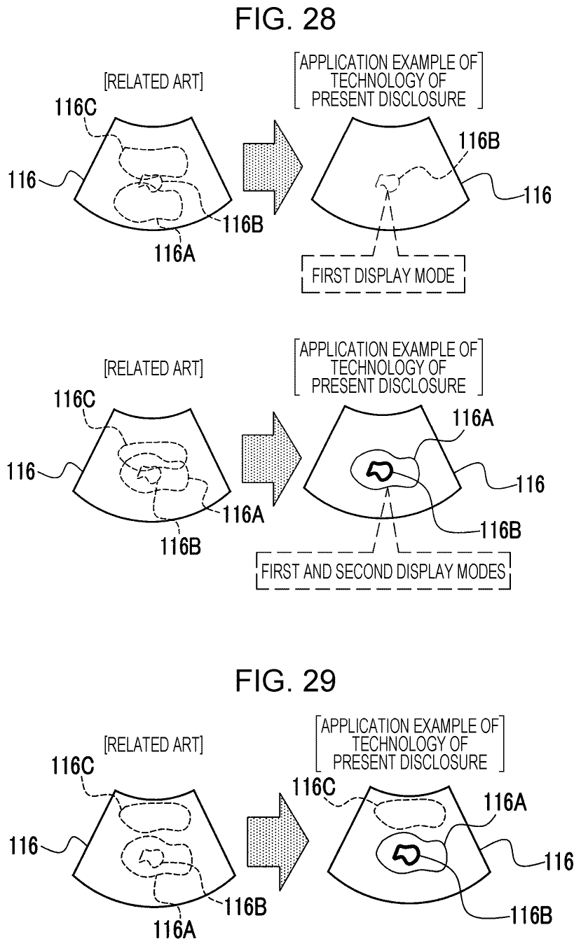

For example, if it is determined that both the overlapping degree 124 between the site region 116A and the lesion region 116B and an overlapping degree 124 between the site region 116C and the lesion region 116B are less than the preset overlapping degree (step ST20: N), as illustrated in FIG. 28 as an example, the ultrasound image 116 is displayed in the first display mode. In an example of the related art, the site regions 116A and 116C are displayed together with the lesion region 116B, so that it is unclear whether the lesion region 116B is relevant to the site region 116A or 116C. In contrast, in the fifth modification, by the execution of step ST22 illustrated in FIG. 27A, the site regions 116A and 116C are not displayed, and the lesion region 116B is displayed, and thus, it is possible to suppress erroneous recognition of a site region having low relevance to the lesion region 116B by a user or the like as a site region having relevance to the lesion region 116B.

In addition, if it is determined that the overlapping degree 124 between the site region 116A and the lesion region 116B is greater than or equal to the preset overlapping degree and the overlapping degree 124 between the site region 116C and the lesion region 116B is less than the preset overlapping degree, as illustrated in FIG. 28 as an example, the site region 116A and the lesion region 116B are displayed in the second display mode, and the site region 116C and the lesion region 116B are displayed in the first display mode. In an example of the related art, the site regions 116A and 116C are displayed together with the lesion region 116B, so that it is unclear whether the lesion region 116B is relevant to the site region 116A or 116C. In contrast, in the fifth modification, since the site region 116C is not displayed and the lesion region 116B is displayed by the execution of step ST22 illustrated in FIG. 27A, it is possible to suppress erroneous recognition of the site region 116C having low relevance to the lesion region 116B by a user or the like as a site region having relevance to the lesion region 116B. In addition, since the site region 116A and the lesion region 116B are displayed in a comparable and distinguishable manner by the execution of step ST24 illustrated in FIG. 27A, a user or the like can recognize that the site region having high relevance to the lesion region 116B is the site region 116A.

Note that the display in a comparable and distinguishable manner is, for example, display in a display mode in which the distinctiveness between the site region 116A and the lesion region 116B is emphasized. The distinctiveness is emphasized by, for example, a color difference and/or a luminance difference between the site region 116A and the lesion region 116B. The color difference herein is, for example, a complementary color relationship in a hue circle. In addition, regarding the luminance difference, for example, if the lesion region 116B is expressed in a luminance range of “150 to 255 gradations”, the site region 116A may be expressed in a luminance range of “0 to 50 gradations”. In addition, the distinctiveness is also emphasized by, for example, differentiating a display mode of a frame (e.g., a circumscribed frame or an outer contour) that surrounds the position of the site region 116A in an identifiable manner from a display mode of a frame (e.g., a circumscribed frame or an outer contour) that surrounds the position of the lesion region 116B in an identifiable manner.

### Sixth Modification

Although the site region 116C is not displayed if it is determined that the overlapping degree 124 between the site region 116C and the lesion region 116B is less than the preset overlapping degree in the fifth modification above, the technology of the present disclosure is not limited to this. For example, the display mode for each of the plurality of site regions may differ depending on a corresponding one of a plurality of positional relationships. The plurality of positional relationships herein are positional relationships between a plurality of site regions for a plurality of types of sites and the lesion region 116B.

For example, as illustrated in FIG. 29, if the overlapping degree 124 between the site region 116A and the lesion region 116B is greater than or equal to the preset overlapping degree, the site region 116A and the lesion region 116B are displayed in the second display mode. In addition, if the overlapping degree 124 between the site region 116C and the lesion region 116B is less than the preset overlapping degree, the site region 116C is displayed on condition that the overlapping degree 124 is “0”. In addition, even if the overlapping degree 124 between the site region 116C and the lesion region 116B is less than the preset overlapping degree, as long as the overlapping degree 124 has a value greater than “0”, the site region 116C is not displayed as in the example illustrated in the lower part of FIG. 28.

Accordingly, it is possible to suppress erroneous recognition of the site region 116C having low relevance to the lesion region 116B by a user or the like as a site region having relevance to the lesion region 116B, and the user or the like can recognize the site region 116A as a site region having high relevance to the lesion region 116B. In addition, if the site region 116C is present at a position not likely to be erroneously recognized by a user or the like as a site region having relevance to the lesion region 116B, since the site region 116C is displayed, the user or the like can grasp the positional relationship between the site region 116A and the site region 116C and the positional relationship between the site region 116C and the lesion region 116B.

Note that on condition that the overlapping degree 124 between the site region 116C and the lesion region 116B is “0”, a display intensity of the site region 116C (e.g., the luminance of the contour and/or the thickness of the contour line of the site region 116C) may be increased as the distance between the site region 116C and the lesion region 116B increases.

In addition, the display mode for each of the plurality of site regions may differ depending on the positional relationship between the plurality of site regions. For example, if the site region 116A overlapping with the lesion region 116B at the preset overlapping degree or more overlaps with the site region 116C at less than the preset overlapping degree, the site region 116C may be hidden, and if the site region 116A overlapping with the lesion region 116B at the preset overlapping degree or more does not overlap with the site region 116C, the site region 116C may be displayed. In addition, if the site region 116A overlapping with the lesion region 116B at the preset overlapping degree or more does not overlap with the site region 116C, the display intensity of the site region 116C may be increased as the distance between the site region 116A and the site region 116C increases.

Thus, it is possible to suppress erroneous recognition of the site region 116C having low relevance to the lesion region 116B by a user or the like as a site region having relevance to the lesion region 116B. In addition, the user or the like can grasp the positional relationship between the site region 116A and the site region 116C.

### Seventh Modification

Although an example of a form has been described in which, in the display control process illustrated in FIG. 27A and FIG. 27B, the overlapping degree 124 between each of the plurality of site regions and the lesion region 116B is calculated, and all the site regions are displayed depending on the calculated overlapping degree 124, the technology of the present disclosure is not limited to this. For example, the overlapping degree 124 between each of the plurality of site regions and the lesion region 116B is calculated, and only a site region having relevance to a maximum overlapping degree among a plurality of calculated overlapping degrees 124 may be displayed.

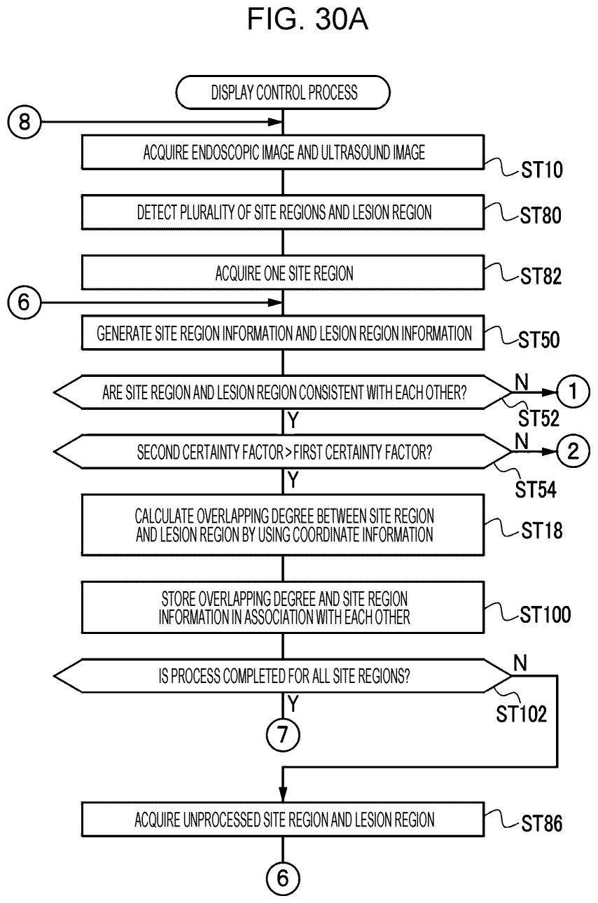

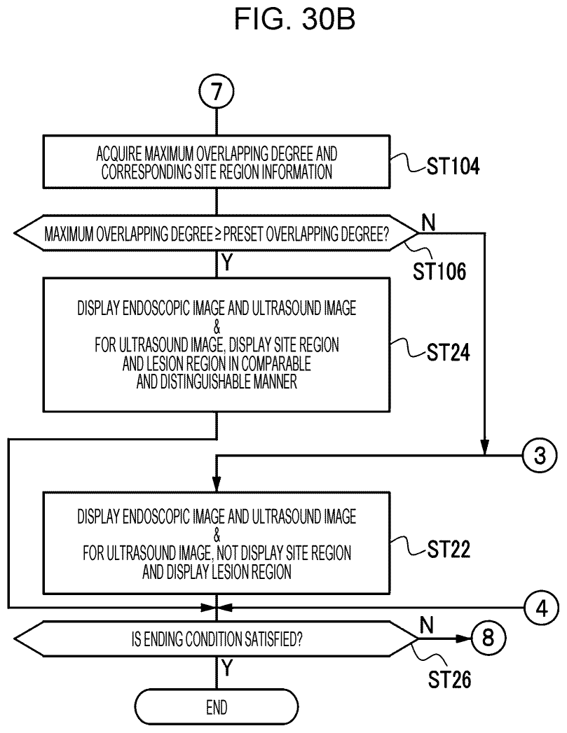

For example, in this case, the processor 104 executes the display control process illustrated in FIG. 30A and FIG. 30B. The flowcharts illustrated in FIG. 30A and FIG. 30B are different from the flowcharts illustrated in FIG. 27A and FIG. 27B in that step ST100 to step ST106 are applied instead of step ST20. Note that the same steps as those in the flowcharts illustrated in FIG. 27A and FIG. 27B are denoted by the same step numbers, and description thereof is omitted herein.

In the display control process illustrated in FIG. 30A, in step ST100, the positional relationship identification unit 104D stores, in the RAM 106, the overlapping degree 124 calculated in step ST18 and the site region information 118 generated in step ST50 in association with each other. In step ST100, the overlapping degree 124 for each of the plurality of site regions (e.g., the site regions 116A and 116C) detected in step ST80 and the site region information 118 are stored in association with each other in the RAM 106. That is, a plurality of overlapping degrees 124 and a plurality of pieces of site region information 118 are stored in the RAM 106 in a one-to-one correspondence. After step ST100 is executed, the display control process proceeds to step ST102.

In step ST102, the positional relationship identification unit 104D determines whether step ST50 and subsequent steps have been executed for all the site regions detected in step ST80. In step ST102, if step ST50 and subsequent steps are yet to be executed for all the site regions detected in step ST80, the determination is negative, and the display control process proceeds to step ST86. In step ST102, if step ST50 and subsequent steps have been executed for all the site regions detected in step ST80, the determination is positive, and the display control process proceeds to step ST104 illustrated in FIG. 30B.

In step ST104 illustrated in FIG. 30B, the positional relationship identification unit 104D acquires, from the RAM 106, the maximum overlapping degree, which is the largest overlapping degree 124 among the plurality of overlapping degrees 124 stored in the RAM 106, and the site region information 118 associated with the maximum overlapping degree. After step ST104 is executed, the display control process proceeds to step ST106.

In step ST106, the positional relationship identification unit 104D determines whether the maximum overlapping degree acquired in step ST104 is greater than or equal to the preset overlapping degree. If the maximum overlapping degree is less than the preset overlapping degree in step ST106, the determination is negative, and the display control process proceeds to step ST22. Then, in step ST22 and subsequent steps, the process using the site region information 118 acquired in step ST104 and the lesion region information 120 generated in step ST50 is executed. On the other hand, if the maximum overlapping degree is greater than or equal to the preset overlapping degree in step ST106, the determination is positive, and the display control process proceeds to step ST24. Then, in step ST24 and subsequent steps, the process using the site region information 118 acquired in step ST104 and the lesion region information 120 generated in step ST50 is executed.

In this manner, by performing the display control process illustrated in FIG. 30A and FIG. 30B, the ultrasound image 116 is displayed in a display mode in accordance with the positional relationship between the lesion region 116B and a maximum site region, which is a site region having the largest overlapping degree with the lesion region 116B, among the plurality of site regions. Accordingly, even if the plurality of site regions are detected, a user or the like can grasp the site region and the lesion region 116B having high relevance to each other.

### Eighth Modification

Although the above seventh modification has described an example of a form in which the ultrasound image 116 is displayed in a display mode in accordance with the positional relationship between the lesion region 116B and the maximum site region among the plurality of site regions, the technology of the present disclosure is not limited to this. For example, the ultrasound image 116 may be displayed in a display mode in accordance with the positional relationship between the lesion region 116B and a site region identified by using the lesion region information 120 and the site region information 118 including the largest first certainty factor 118C among a plurality of first certainty factors 118C acquired from a plurality of pieces of site region information 118.

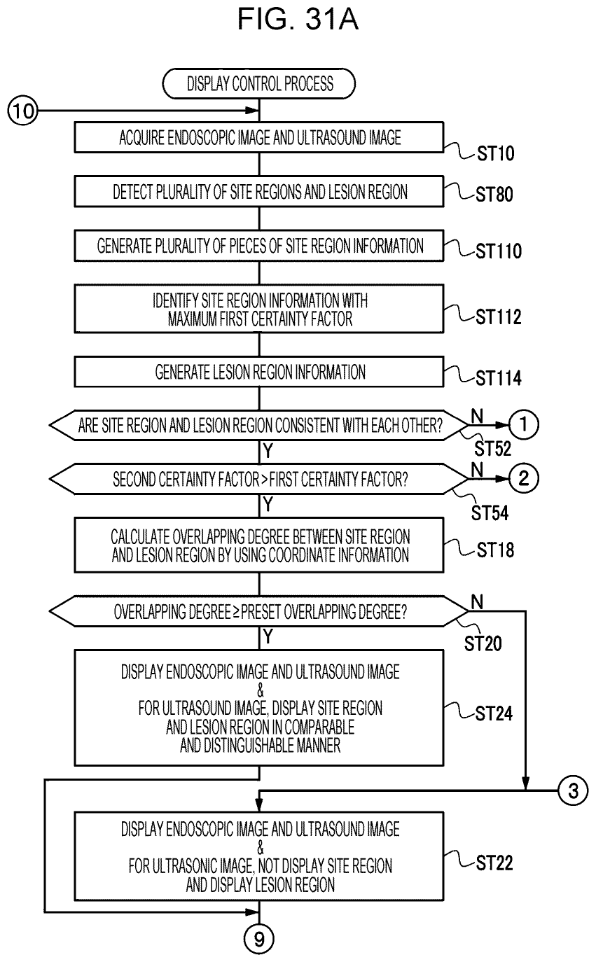

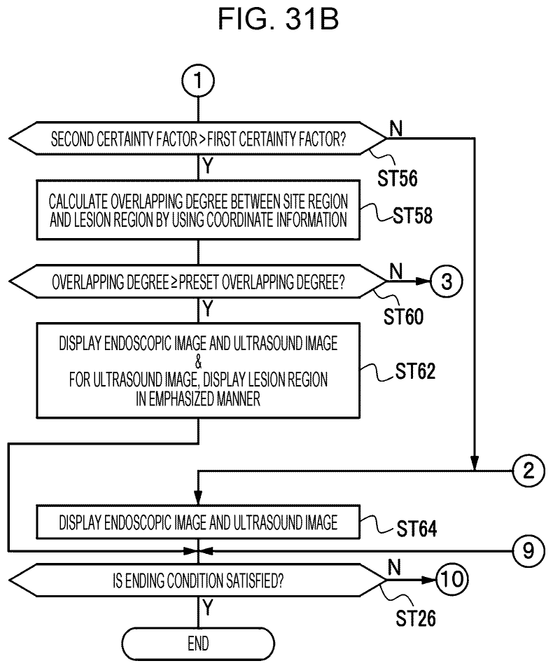

For example, in this case, the processor 104 executes the display control process illustrated in FIG. 31A and FIG. 31B. The flowcharts illustrated in FIG. 31A and FIG. 31B are different from the flowcharts illustrated in FIG. 27A and FIG. 27B in that step ST110 to step ST114 are applied instead of step ST82 and step ST50. Note that the same steps as those in the flowcharts illustrated in FIG. 27A and FIG. 27B are denoted by the same step numbers, and description thereof is omitted herein.

In the display control process illustrated in FIG. 31A, in step ST110, the detection unit 104B generates a plurality of pieces of site region information 118 related to the plurality of site regions (e.g., the site regions 116A and 116C) detected in step ST80. After step ST110 is executed, the display control process proceeds to step ST112.

In step ST112, the detection unit 104B compares a plurality of first certainty factors 118C included in the plurality of pieces of site region information 118 generated in step ST110 with one another to identify a piece of site region information 118 including the largest first certainty factor 118C from the plurality of pieces of site region information 118 generated in step ST110. In step ST52 and subsequent steps, the process using the piece of site region information 118 identified in step ST112 is executed. After step ST112 is executed, the display control process proceeds to step ST114.

In step ST114, the detection unit 104B generates the lesion region information 120 related to the lesion region 116B detected in step ST80. In step ST52 and subsequent steps, the process using the lesion region information 120 generated in step ST114 is executed.

Note that after step ST22 illustrated in FIG. 31A is executed, the display control process proceeds to step ST26 illustrated in FIG. 31B. In addition, if the determination is negative in step ST26 illustrated in FIG. 31B, the process proceeds to step ST10 illustrated in FIG. 31A, and if the determination is positive in step ST26, the display control process ends.

In this manner, by performing the display control process illustrated in FIG. 31A and FIG. 31B, the ultrasound image 116 is displayed in a display mode in accordance with the positional relationship between the lesion region 116B and the site region identified by using the lesion region information 120 and the site region information 118 including the largest first certainty factor 118C among the plurality of first certainty factors 118C acquired from the plurality of pieces of site region information 118. Accordingly, even if the plurality of site regions are detected, a user or the like can grasp the site region and the lesion region 116B having high relevance to each other.

### Ninth Modification

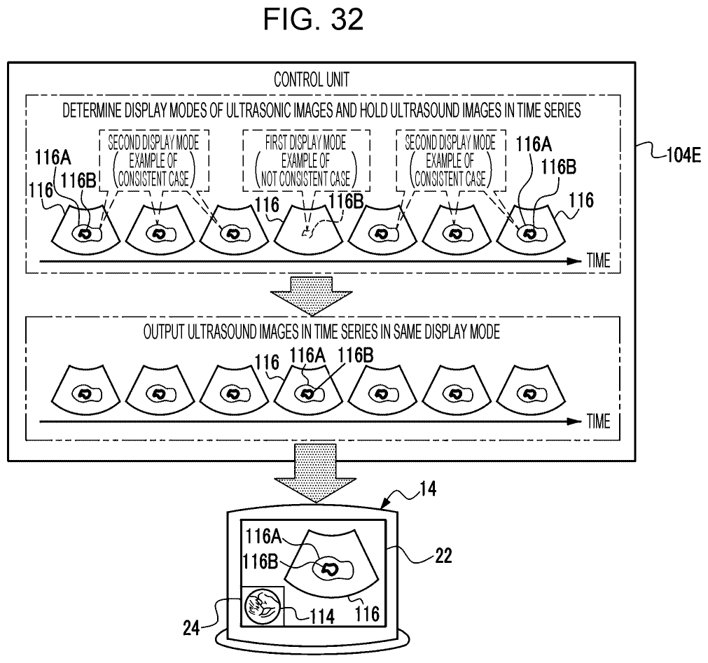

The above embodiment has described an example of a form in which the display control process is executed on the plurality of ultrasound images 116 included in the ultrasound moving image 26 frame by frame, and thus, the ultrasound image 116 is displayed in the display mode determined for each frame. In this case, as illustrated in FIG. 32 as an example, if the display control process is executed on the plurality of ultrasound images 116 in time series, the display mode of the ultrasound image 116 may differ between a case where the site region 116A and the lesion region 116B are consistent with each other and a case where the site region 116A and the lesion region 116B are not consistent with each other. For example, if the site region 116A and the lesion region 116B are not consistent with each other, the ultrasound image 116 may be displayed in the first display mode, and if the site region 116A and the lesion region 116B are consistent with each other, the ultrasound image 116 may be displayed in the second display mode.

In this case, if ultrasound images 116 of several frames, which are adjacent to and precede and follow, in time series, an ultrasound image 116 displayed in the first display mode are displayed in the second display mode, there is a high possibility that the ultrasound image 116 displayed in the first display mode is supposed to be displayed in the second display mode. That is, although the site region 116A and the lesion region 116B are consistent with each other, since the determination unit 104C determines that the site region 116A and the lesion region 116B are not consistent with each other, it is likely that the ultrasound image 116 is displayed in the first display mode.

Thus, in the ultrasonic endoscope system 10 according to the ninth modification, the control unit 104E corrects the display mode of an ultrasound image 116 that may have been erroneously determined by the determination unit 104C, based on the display mode of an ultrasound image 116 that has been correctly determined by the determination unit 104C. The display mode of the ultrasound image 116 that may have been erroneously determined is a display mode corresponding to the ultrasound image 116 used as a determination target in a case where the determination unit 104C determines that the combination of the site region 116A and the lesion region 116B is not correct (i.e., not consistent with each other). In addition, the display mode of the ultrasound image 116 that has been correctly determined is a display mode (i.e., a display mode determined in the same manner as in the above embodiment) corresponding to the ultrasound image 116 that is a determination target in a case where the determination unit 104C determines that the combination of the site region 116A and the lesion region 116B is correct (i.e., consistent with each other).

In the example illustrated in FIG. 32, the control unit 104E determines the display mode for each of the plurality of ultrasound images 116 in the manner described in the above embodiment, and holds the plurality of ultrasound images 116 in time series in the order in which the display mode is determined. The control unit 104E holds the plurality of ultrasound images 116 by a FIFO method. That is, each time a new frame is added, the control unit 104E outputs the oldest frame to the display apparatus 14. In the example illustrated in FIG. 32, for convenience of illustration, the control unit 104E holds the ultrasound images 116 of a first frame to a seventh frame in time series.

It is decided that each of the ultrasound images 116 of the first frame to the third frame and the fifth frame to the seventh frame is to be displayed in the second display mode as a result of the determination unit 104C determining that the combination of the site region 116A and the lesion region 116B is correct. It is decided that the ultrasound image 116 of the fourth frame is to be displayed in the first display mode as a result of the determination unit 104C determining that the combination of the site region 116A and the lesion region 116B is not correct.

Although the combination of the site region 116A and the lesion region 116B is determined to be correct for each of the ultrasound images 116 of three frames, which precede and follow, in time series, the ultrasound image 116 for which the combination of the site region 116A and the lesion region 116B is determined to be not correct (i.e., the ultrasound image 116 of the fourth frame) herein, this is merely an example. The ultrasound images 116 of four or more frames for which the combination of the site region 116A and the lesion region 116B is determined to be correct may be adjacent to and precede and follow, in time series, the ultrasound image 116 for which the combination of the site region 116A and the lesion region 116B is determined to be not correct.

In addition, although the ultrasound image 116 for which the combination of the site region 116A and the lesion region 116B is determined to be not correct is the ultrasound image 116 of one frame herein, this is merely an example. For example, the number of frames of the ultrasound images 116 for which the combination of the site region 116A and the lesion region 116B is determined to be not correct may be sufficiently smaller than the number of frames of the ultrasound images 116 for which the combination of the site region 116A and the lesion region 116B is determined to be correct. For example, the sufficiently small number of frames is the number of frames that is about several tenths to several hundredths of the number of frames of the ultrasound image 116 for which the combination of the site region 116A and the lesion region 116B is determined to be correct. The sufficiently small number of frames may be a fixed value or may be a variable value that is changed in accordance with an instruction received by the reception apparatus 62 and/or various conditions.

In the example illustrated in FIG. 32, the control unit 104E corrects the display mode of the ultrasound image 116 of the fourth frame with reference to the display mode determined for the ultrasound images 116 of the first frame to the third frame and the fifth frame to the seventh frame among the plurality of ultrasound images 116 held in time series. In the example illustrated in FIG. 32, since the second display mode is determined for the ultrasound images 116 of the first frame to the third frame and the fifth frame to the seventh frame, the first display mode determined for the ultrasound image 116 of the fourth frame is corrected to the second display mode. Accordingly, the display mode of all the ultrasound images 116 held in time series is made uniform to the second display mode. The control unit 104E outputs the ultrasound images 116 in time series to the display apparatus 14 by making the display mode of the ultrasound images 116 of the first frame to the seventh frame uniform to the second display mode, thereby displaying the ultrasound images 116 on the first screen 22. Accordingly, a user or the like can be prevented from erroneously recognizing that the combination of the site region 116A and the lesion region 116B is not correct, although the combination of the site region 116A and the lesion region 116B is correct.

### Tenth Modification

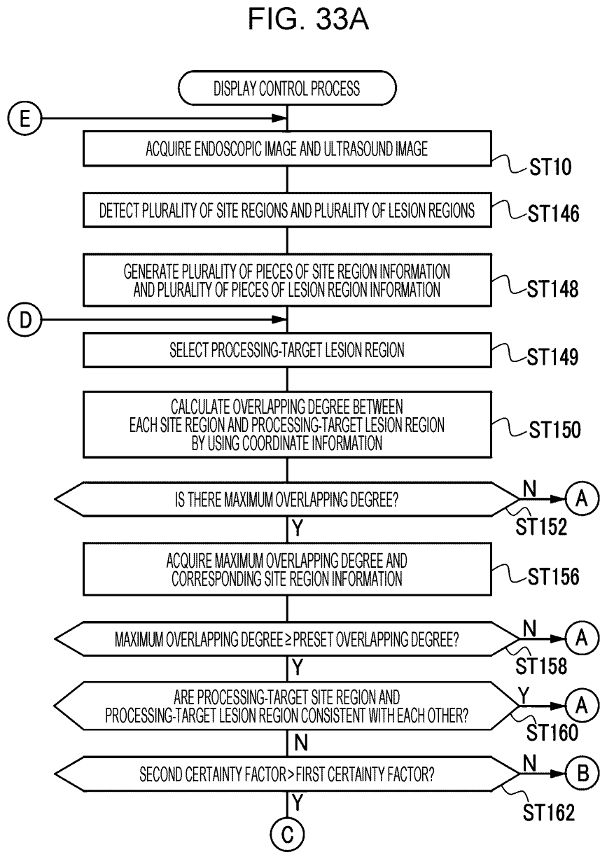

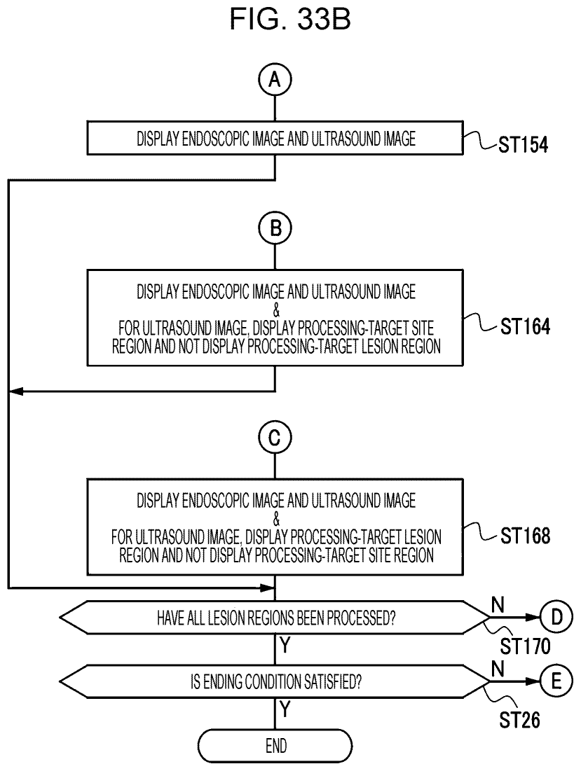

The tenth modification will describe, with reference to FIG. 33A and FIG. 33B, a display control process of an algorithm different from that of the display control process (see FIG. 31A and FIG. 31B) described in the eighth modification above. The flowcharts illustrated in FIG. 33A and FIG. 33B are different from the flowcharts illustrated in FIG. 31A and FIG. 31B in that step ST146 to step ST170 are applied instead of step ST80 to step ST64. Only processing different from that in the flowcharts illustrated in FIG. 31A and FIG. 31B will be described in the tenth modification.

In step ST146 illustrated in FIG. 33A, the detection unit 104B performs AI image recognition processing to detect a plurality of site regions (e.g., a pancreas and a kidney) and a plurality of lesion regions 116B (e.g., pancreatic cancer and kidney cancer) from the ultrasound image 116. After step ST146 is executed, the display control process proceeds to step ST148.

In step ST148, the detection unit 104B generates a plurality of pieces of site region information 118 corresponding to the plurality of site regions detected in step ST146. In addition, the detection unit 104B generates a plurality of pieces of lesion region information 120 corresponding to the plurality of lesion regions 116B detected in step ST146. After step ST148 is executed, the display control process proceeds to step ST149.

In step ST149, the positional relationship identification unit 104D selects, from among the plurality of lesion regions 116B detected in step ST146, a processing-target lesion region that is one lesion region 116B not subjected to the processing in step ST150 and subsequent steps. After step ST149 is executed, the display control process proceeds to step ST150.

In step ST150, the positional relationship identification unit 104D acquires the coordinate information 118A from each of the plurality of pieces of site region information 118 generated in step ST148. In addition, the positional relationship identification unit 104D acquires the coordinate information 120A from the lesion region information 120 corresponding to the processing-target lesion region selected in step ST149 from among the plurality of pieces of lesion region information 120 generated in step ST148. In addition, the positional relationship identification unit 104D calculates the overlapping degree 124 between each of the site regions detected in step ST146 and the processing-target lesion region. For example, if the plurality of site regions are the pancreas and the kidney, the overlapping degree 124 for the pancreas and the processing-target lesion region and the overlapping degree 124 for the kidney and the processing-target lesion region are calculated. Thus, in step ST150, the plurality of overlapping degrees 124 are calculated. After step ST150 is executed, the display control process proceeds to step ST152.

In step ST152, the positional relationship identification unit 104D determines whether the maximum overlapping degree 124 is present among the plurality of overlapping degrees 124 calculated in step ST150. If the maximum overlapping degree 124 is not present among the plurality of overlapping degrees 124 in step ST152, the determination is negative, and the display control process proceeds to step ST154 illustrated in FIG. 33B. If the maximum overlapping degree 124 is present among the plurality of overlapping degrees 124 in step ST152, the determination is positive, and the display control process proceeds to step ST156.

Note that, although it is determined herein whether the maximum overlapping degree 124 is present, the technology of the present disclosure is not limited to this, and it may be determined whether the overlapping degree 124 greater than or equal to a certain reference value is present.

In step ST154 illustrated in FIG. 33B, the control unit 104E displays the ultrasound image 116 acquired in step ST10 on the first screen 22 and displays the endoscopic image 114 acquired in step ST10 on the second screen 24. After step ST154 is executed, the display control process proceeds to step ST170.

In step ST156 illustrated in FIG. 33A, the positional relationship identification unit 104D acquires the maximum overlapping degree, which is the largest overlapping degree 124 among the plurality of overlapping degrees 124 calculated in step ST150, and the site region information 118 associated with the maximum overlapping degree. After step ST156 is executed, the display control process proceeds to step ST158.

In step ST158, the positional relationship identification unit 104D determines whether the maximum overlapping degree acquired in step ST156 is greater than or equal to the preset overlapping degree. If the maximum overlapping degree is less than the preset overlapping degree in step ST158, the determination is negative, and the display control process proceeds to step ST154 illustrated in FIG. 33B. If the maximum overlapping degree is greater than or equal to the preset overlapping degree in step ST158, the determination is positive, and the display control process proceeds to step ST160.

In step ST160, in the same manner as that in step ST16 illustrated in FIG. 14, the determination unit 104C determines whether a processing-target site region and the processing-target lesion region are consistent with each other. The processing-target site region herein is a site region corresponding to the site region information 118 acquired in step ST156 (i.e., a site region identified from the site region information 118 acquired in step ST156). The determination of whether the processing-target site region and the processing-target lesion region are consistent with each other is performed by using the site region information 118 acquired in step ST156 and the lesion region information 120 corresponding to the processing-target lesion region selected in step ST149. The lesion region information 120 corresponding to the processing-target lesion region selected in step ST149 is the lesion region information 120 corresponding to the processing-target lesion region selected in step ST149 from among the plurality of pieces of lesion region information 120 generated in step ST148.

If the processing-target site region and the processing-target lesion region are consistent with each other step ST160, the determination is positive, and the process proceeds to step ST154 illustrated in FIG. 33B. If the processing-target site region and the processing-target lesion region are not consistent with each other in step ST160, the determination is negative, and the process proceeds to step ST162.

In step ST162, the positional relationship identification unit 104D acquires the first certainty factor 118C from the site region information 118 acquired in step ST156. In addition, the positional relationship identification unit 104D acquires the lesion region information 120 corresponding to the processing-target lesion region selected in step ST149 from among the plurality of pieces of lesion region information 120 generated in step ST148, and acquires the second certainty factor 120C from the acquired lesion region information 120. Then, the positional relationship identification unit 104D determines whether the second certainty factor 120C is greater than the first certainty factor 118C. If the second certainty factor 120C is less than or equal to the first certainty factor 118C in step ST162, the determination is negative, and the process proceeds to step ST164 illustrated in FIG. 33B. If the second certainty factor 120C is greater than the first certainty factor 118C in step ST162, the determination is positive, and the display control process proceeds to step ST168 illustrated in FIG. 33B.

Although it is determined whether the magnitude relationship “first certainty factor 118C >second certainty factor 120C” is satisfied herein, the technology of the present disclosure is not limited to this. For example, it may be determined whether the difference between the first certainty factor 118C and the second certainty factor 120C exceeds a threshold value.

In addition, the condition in a case of comparing the first certainty factor 118C and the second certainty factor 120C with each other may be changed depending on the type of the processing-target site region. For example, a different threshold value may be provided in accordance with the type of the processing-target site region, and the first certainty factor 118C and the second certainty factor 120C exceeding the threshold value may be compared with each other.

In step ST164 illustrated in FIG. 33B, the control unit 104E displays the ultrasound image 116 acquired in step ST10 on the first screen 22 and displays the endoscopic image 114 acquired in step ST10 on the second screen 24. In this case, the control unit 104E displays the processing-target site region in the ultrasound image 116 and does not display the processing-target lesion region in the ultrasound image 116. For example, in a case where the processing-target lesion region is a region indicating the pancreatic cancer and the processing-target site region is a region indicating the kidney, the region indicating the kidney is displayed, and the region indicating the pancreatic cancer is not displayed.

Note that, in this specification, the concept of non-display includes, in addition to a state of not being completely displayed, a state in which the display intensity (e.g., luminance and/or concentration) is reduced to a perception level (e.g., a perception level known in advance by a sensory test using an actual machine and/or computer simulation) to an extent that is not erroneously distinguished by the doctor 16. After step ST164 is executed, the display control process proceeds to step ST170.

In step ST168 illustrated in FIG. 33B, the control unit 104E displays the ultrasound image 116 acquired in step ST10 on the first screen 22 and displays the endoscopic image 114 acquired in step ST10 on the second screen 24. In this case, the control unit 104E displays the ultrasound image 116, which is displayed on the first screen 22, in the first display mode. That is, the control unit 104E displays the processing-target lesion region in the ultrasound image 116 and does not display the processing-target site region in the ultrasound image 116. For example, in a case where the processing-target lesion region is a region indicating the pancreatic cancer and the processing-target site region is a region indicating the kidney, the region indicating the pancreatic cancer is displayed, and the region indicating the kidney is not displayed. After step ST168 is executed, the display control process proceeds to step ST170.

Note that in step ST154, step ST164, and/or step ST168, whether the processing-target site region is to be finally displayed or not displayed may be determined in accordance with the type of the processing-target lesion region and/or the type of the processing-target site region.

In addition, a site region indicating a specific organ (e.g., the kidney 66 in a scene of examining the pancreas 65) is preferably not displayed at all times. However, even if the site region indicating the specific organ is not displayed at all times, the site region indicating the specific organ is used in processing related to the overlapping degree 124 and processing related to the determination of the magnitude relationship between the first certainty factor 118C and the second certainty factor 120C (i.e., used in step ST150 to step ST162).

In step ST170, the positional relationship identification unit 104D determines whether all the plurality of lesion regions 116B detected in step ST146 have been used in step ST150 and subsequent steps. In step ST170, if all the plurality of lesion regions 116B detected in step ST146 have not been used in step ST150 and subsequent steps, the determination is negative, and the display control process proceeds to step ST149 illustrated in FIG. 33A. In step ST170, if all the plurality of lesion regions 116B detected in step ST146 have been used in step ST150 and subsequent steps, the determination is positive, and the display control process proceeds to step ST26.

As described above, in the tenth modification, the certainty of the processing-target lesion region is determined in accordance with the positional relationship between the processing-target lesion region and the processing-target site region and the relationship between the first certainty factor 118C and the second certainty factor 120C. That is, by performing step ST149 to step ST162 illustrated in FIG. 33A, the certainty of the processing-target lesion region is determined. Then, the determination result is displayed on the first screen 22 (see step ST164 and step ST168). Accordingly, it is possible to prevent a processing-target site region that is not correctly combined with the processing-target lesion region from being displayed, and to prevent a processing-target lesion region that is not correctly combined with the processing-target site region from being displayed. As a result, it is possible to prevent a user or the like from performing erroneous differentiation.

In addition, in the tenth modification, if the positional relationship between the processing-target lesion region and the processing-target site region is a preset positional relationship (e.g., step ST158: Y), the processing-target site region and the processing-target lesion region are not consistent with each other (e.g., step ST160: N), and the relationship between the first certainty factor 118C and the second certainty factor 120C is a preset certainty factor relationship (e.g., step ST162: Y), it is determined that the processing-target lesion region is certain. That is, if the determination is positive in step ST162 as a result of step ST156 to step ST162 illustrated in FIG. 33A, it is determined that the processing-target lesion region is certain. If it is determined that the processing-target lesion region is certain, the determination result is displayed on the first screen 22 (see step ST168). Thus, the processing-target lesion region determined to be certain is displayed. As a result, a user or the like can grasp the processing-target lesion region determined to be certain.

In addition, in the tenth modification, the certainty of the processing-target site region is determined. That is, by step ST149 to step ST162 illustrated in FIG. 33A, the certainty of the processing-target site region is determined. Then, the determination result is displayed on the first screen 22 (see step ST164 and step ST168). Accordingly, it is possible to prevent a processing-target site region that is not correctly combined with the processing-target lesion region from being displayed, and to prevent a processing-target lesion region that is not correctly combined with the processing-target site region from being displayed. As a result, it is possible to prevent a user or the like from performing erroneous differentiation.

Note that, if it is determined that the processing-target lesion region is certain, information indicating that the detection of the lesion may be displayed on the display apparatus 14. In this case, for example, the ultrasound image 116 is displayed on the first screen 22 in the third display mode (see FIG. 20). Accordingly, a user or the like can visually recognize the position of the processing-target lesion region on the first screen 22.

### Other Modifications

Although the display mode of the site region 116A and the lesion region 116B is determined for each frame in the above embodiment, the technology of the present disclosure is not limited to this. For example, the display mode of the site region 116A and the lesion region 116B may be determined for each preset number of frames (e.g., for each several frames or for each several tens of frames). In this case, since the number of times of performing the display control process is reduced, it is possible to reduce a load applied to the processor 104 as compared with a case where the display control process is performed for each frame. In addition, in a case where the display mode of the site region 116A and the lesion region 116B is determined for each preset number of frames in this manner, the display mode of the site region 116A and the lesion region 116B may be determined at frame intervals at which the display mode (e.g., the first to third display modes) are visually perceived due to an afterimage phenomenon.

Although the above embodiment has described an example of a form in which the contour of the site region 116A and the contour of the lesion region 116B are displayed, the technology of the present disclosure is not limited to this. For example, the positions of the site region 116A and the lesion region 116B may be displayed so as to be identifiable by bounding boxes used in the AI image recognition processing. In this case, the control unit 104E may cause the display apparatus 14 to display a bounding box for identifying the site region 116A and a bounding box for identifying the lesion region 116B in substantially the same display mode as the display mode applied to the site region 116A and the lesion region 116B. For example, display and non-display of the bounding box for identifying the site region 116A may be switched, the display mode of the contour or the like of the bounding box for identifying the site region 116A may be changed under the same conditions as those in the above embodiment, or the display mode of the contour or the like of the bounding box for identifying the lesion region 116B may be changed under the same conditions as those in the above embodiment.

In addition, the positional relationship identification unit 104D may calculate the overlapping degree 124 and/or the distance 126 by using the bounding box for identifying the site region 116A and the bounding box for identifying the lesion region 116B. An example of the overlapping degree 124 in this case is IoU using the bounding box for identifying the site region 116A and the bounding box for identifying the lesion region 116B. The IoU in this case is a ratio of the area in which the bounding box for identifying the site region 116A overlaps with the bounding box for identifying the lesion region 116B to the area of a region in which the bounding box for identifying the site region 116A is combined with the bounding box for identifying the lesion region 116B. The overlapping degree 124 may also be a ratio of the area in which the bounding box for identifying the site region 116A overlaps with the bounding box for identifying the lesion region 116B to the total area of the bounding box for identifying the lesion region 116B. In addition, an example of the distance 126 in this case is a distance between the bounding box for identifying the site region 116A and part of a contour of a region not overlapping with the bounding box for identifying the site region 116A in the bounding box for identifying the lesion region 116B. For example, the part of the contour of the region not overlapping with the bounding box for identifying the site region 116A is a position farthest from the bounding box for identifying the site region 116A in the contour of the region not overlapping with the site region 116A.

Although the above embodiment has described an example of a form in which the site regions 116A and 116C are not displayed in the first display mode, this is merely an example, and the display intensity of the site regions 116A and/or 116C may be made lower than the display intensity of the lesion region 116B instead of not displaying the site regions 116A and/or 116C.

Although the above embodiment has described an example of a form in which, if the second certainty factor 120C is greater than the first certainty factor 118C, the ultrasound image 116 is displayed in any of the first to third display modes, the technology of the present disclosure is not limited to this. For example, if the first certainty factor 118C is greater than the second certainty factor 120C, the site region 116A or 116C and the lesion region 116B may be displayed in a comparable manner on condition that the overlapping degree 124 is less than the preset overlapping degree or the overlapping degree 124 is “0”. In addition, the site region 116A or 116C and the lesion region 116B may be displayed in the second display mode.

Although the above embodiment has described an example of a form in which the intensity of the contour of the site region 116A is set to an intensity in accordance with the overlapping degree 124, the technology of the present disclosure is not limited to this. If the overlapping degree 124 is greater than or equal to the preset overlapping degree, the display intensities of both the site region 116A and the lesion region 116B may be increased as the overlapping degree 124 is larger.

Although the above embodiment has described an example of a form in which the ultrasound image 116 is displayed on the first screen 22, this is merely an example. For example, the ultrasound image 116 may be displayed on the entire screen of the display apparatus 14.

Although the processor 104 directly acts on the display apparatus 14 to cause the display apparatus 14 to display the ultrasound image 116 in the above embodiment, this is merely an example. For example, the processor 104 may indirectly act on the display apparatus 14 to cause the display apparatus 14 to display the ultrasound image 116. For example, in this case, screen information indicating a screen (e.g., at least the first screen 22 out of the first screen 22 and the second screen 24) to be displayed on the display apparatus 14 is temporarily stored in an external storage (omitted from illustration). Then, the processor 104 or a processor other than the processor 104 acquires the screen information from the external storage, and, based on the acquired screen information, causes the display apparatus 14 or a display apparatus other than the display apparatus 14 to display at least the first screen 22 out of the first screen 22 and the second screen 24.

As a specific example in this case, there is an example of a form in which the processor 104 causes the display apparatus 14 or a display apparatus other than the display apparatus 14 to display at least the first screen 22 out of the first screen 22 and the second screen 24 by using cloud computing.

Although the above embodiment has described an example of a form in which the display control process is executed on the ultrasound moving image 26, the display control process may be executed on an ultrasound still image.

Although the above embodiment has described an example of a form in which the display control process is executed on the ultrasound image 116 acquired by the ultrasonic endoscope apparatus 12, the technology of the present disclosure is not limited to this. For example, the display control process may be executed on an ultrasound image acquired by an external ultrasound diagnostic apparatus using an external ultrasound probe. The display control process may be executed on a medical image obtained by capturing an image of the observation target region of the examinee 20 by various modalities such as an X-ray diagnostic apparatus, a CT diagnostic apparatus, and/or an MRI diagnostic apparatus. Note that the external ultrasound diagnostic apparatus, the X-ray diagnostic apparatus, the CT diagnostic apparatus, and/or the MRI diagnostic apparatus are/is an example of an “imaging apparatus” according to the technology of the present disclosure.

Although the above embodiment has described an example of a form in which the processor 104 of the display control apparatus 60 performs the display control process, the technology of the present disclosure is not limited to this. For example, a device that performs the display control process may be provided outside the display control apparatus 60. Examples of the device provided outside the display control apparatus 60 include the endoscope processing apparatus 54 and/or the ultrasound processing apparatus 58. Another example of the device provided outside the display control apparatus 60 is a server. For example, the server is implemented by cloud computing. Although cloud computing is given as an example herein, this is merely an example, and for example, the server may be implemented by a mainframe, or may be implemented by network computing such as fog computing, edge computing, or grid computing. The server is merely an example, and at least one personal computer or the like may be used instead of the server. In addition, the display control process may be performed in a distributed manner by a plurality of devices including the display control apparatus 60 and at least one device provided outside the display control apparatus 60.

In addition, although the above embodiment has described an example of a form in which the display control process program 112 is stored in the NVM 108, the technology of the present disclosure is not limited to this. For example, the display control process program 112 may be stored in a portable storage medium such as an SSD or a USB memory. The storage medium is a non-transitory computer-readable storage medium. The display control process program 112 stored in the storage medium is installed in the computer 100 of the display control apparatus 60. The processor 104 executes the display control process in accordance with the display control process program 112.

Although the above embodiment gives the computer 100 as an example, the technology of the present disclosure is not limited to this, and a device including an ASIC, an FPGA, and/or a PLD may be applied instead of the computer 100. Instead of the computer 100, a combination of a hardware configuration and a software configuration may be used.

As a hardware resource for executing the display control process described in the above embodiment, any of the following various processors can be used. Examples of the processors include a processor that is a general-purpose processor functioning as a hardware resource for executing the display control process by executing software, that is, a program. In addition, examples of the processors include a dedicated electronic circuit that is a processor having a circuit configuration specifically designed to execute specific processing, such as an FPGA, a PLD, or an ASIC. A memory is incorporated in or connected to each of the processors, and each of the processors executes the display control process by using the memory.

The hardware resource for executing the display control process may be constituted by one of these various processors, or may be constituted by two or more processors of the same type or different types in combination (e.g., a combination of a plurality of FPGAs, or a combination of a processor and an FPGA). In addition, the hardware resource for executing the display control process may be one processor.

As a first example of a configuration by one processor, one processor may be constituted by a combination of one or more processors and software, and this processor may function as a hardware resource for executing the display control process. As a second example, a processor may be used that implements the functions of the entire system including a plurality of hardware resources for executing the display control process with one integrated circuit (IC) chip as typified by an SoC or the like. In this manner, the display control process is implemented by using one or more of the above various processors as hardware resources.

Furthermore, the hardware configuration of these various processors may be, more specifically, electronic circuitry constituted by combining circuit elements such as semiconductor elements. In addition, the above-described display control process is merely an example. Therefore, it is needless to say that unnecessary steps may be deleted, new steps may be added, or the processing order may be changed without departing from the gist.

The content described above and illustrated in the drawings is a detailed description of portions related to the technology of the present disclosure, and is merely an example of the technology of the present disclosure. For example, the above description regarding the configuration, the function, the operation, and the effect is a description regarding an example of the configuration, the function, the operation, and the effect of the portions related to the technology of the present disclosure. Therefore, it is needless to say that unnecessary portions may be deleted, new elements may be added, or replacement may be performed with respect to the content described above and illustrated in the drawings without departing from the gist of the technology of the present disclosure. In addition, in order to avoid complexity and to facilitate understanding of portions related to the technology of the present disclosure, description of common technical knowledge and the like that do not particularly require description in order to enable implementation of the technology of the present disclosure is omitted from the content described above and illustrated in the drawings.

In the present specification, “A and/or B” is synonymous with “at least one of A and B”. That is, “A and/or B” may mean A alone, B alone, or A and B in combination. In addition, in the present specification, when three or more matters are combined and expressed by “and/or”, the same concept as that of “A and/or B” is applied.

All documents, patent applications, and technical standards described in the present specification are incorporated herein by reference to the same extent as if individual document, patent application, or technical standard is specifically and individually indicated to be incorporated by reference.

In relation to the above embodiment, the following supplementary notes are further disclosed.

### Supplementary Note 1

An image processing apparatus including a processor, in which
the processor is configured to:
detect a first image region and a second image region from a medical image obtained by capturing an image of an observation target region including a site of a human body and a lesion, the first image region indicating the site, the second image region indicating the lesion; and
cause a display apparatus to display a result of detection of the first image region and the second image region in a display mode in accordance with a positional relationship between the first image region and the second image region.

### Supplementary Note 2

The image processing apparatus according to Supplementary Note 1, in which
the processor is configured to acquire a first certainty factor and a second certainty factor, the first certainty factor being a certainty factor for the result of detection of the first image region, the second certainty factor being a certainty factor for the result of detection of the second image region,
the display mode is determined in accordance with the first certainty factor, the second certainty factor, and the positional relationship, and
if the first certainty factor is greater than the second certainty factor and the first image region and the second image region do not overlap with each other, the display mode is a mode in which the first image region and the second image region are displayed so as to be comparable with each other in the medical image.

### Supplementary Note 3

The image processing apparatus according to Supplementary Note 1, in which
the processor is configured to determine whether a combination of the first image region and the second image region is correct based on a correspondence relationship between a plurality of types of the sites and a lesion corresponding to each of the sites, and
if the processor determines that the combination of the first image region and the second image region is correct, the first certainty factor is greater than the second certainty factor, and the first image region and the second image region do not overlap with each other, the display mode is a mode in which the first image region and the second image region are displayed so as to be comparable with each other in the medical image.

### Supplementary Note 4

The image processing apparatus according to Supplementary Note 2 or 3, in which a display intensity of the first image region is determined in accordance with a distance between the first image region and the second image region.

### Supplementary Note 5

The image processing apparatus according to Supplementary Note 1, in which
the observation target region includes a plurality of types of the sites and the lesion, the processor is configured to detect a plurality of the first image regions indicating the plurality of types of the sites and the second image region from the medical image, and
if the second image region overlaps with the plurality of the first image regions in the medical image, the positional relationship is a relationship between a position of a maximally overlapping image region having a largest overlapping degree with the second image region among the plurality of the first image regions and a position of the second image region.

### Supplementary Note 6

The image processing apparatus according to Supplementary Note 1, in which
the processor is configured to acquire a first certainty factor and a second certainty factor, the first certainty factor being a certainty factor for the result of detection of the first image region, the second certainty factor being a certainty factor for the result of detection of the second image region,
the display mode is determined in accordance with the first certainty factor, the second certainty factor, and the positional relationship,
the observation target region includes a plurality of types of the sites and the lesion,
the processor is configured to detect a plurality of the first image regions indicating the plurality of types of the sites and the second image region from the medical image, and
if the second image region overlaps with the plurality of the first image regions in the medical image, the first certainty factor is a largest certainty factor among a plurality of certainty factors for a plurality of results of detection of the plurality of the first image regions.

### Supplementary Note 7

The image processing apparatus according to Supplementary Note 1, in which
the processor is configured to acquire a first certainty factor that is a certainty factor for the result of detection of the first image region, and the display mode is determined in accordance with the first certainty factor and the positional relationship.

### Supplementary Note 8

The image processing apparatus according to Supplementary Note 1, in which
the processor is configured to acquire a second certainty factor that is a certainty factor for the result of detection of the second image region, and the display mode is determined in accordance with the second certainty factor and the positional relationship.

What is claimed is:

1. An image processing apparatus comprising:
a processor, wherein
the processor is configured to:
detect a first image region and a second image region from a medical image obtained by imaging an observation target region including a site of a human body and a lesion, the first image region indicating the site, the second image region indicating the lesion; and
cause a display apparatus to display a result of detection of the first image region and the second image region in a display mode in accordance with a positional relationship between the first image region and the second image region,
the display mode is determined in accordance with the positional relationship and consistency between the site and the lesion.

2. The image processing apparatus according to claim 1, wherein
the display mode for the first image region differs depending on the site, the lesion, and the positional relationship, and
the display mode for the second image region is a mode in which the second image region is displayed on the display apparatus.

3. The image processing apparatus according to claim 2, wherein
in a case in which the site and the lesion are not consistent with each other,
the display mode for the first image region is a mode in which the first image region is not displayed on the display apparatus, and
the display mode for the second image region is a mode in which the second image region is displayed on the display apparatus.

4. The image processing apparatus according to claim 2, wherein
in a case in which the site and the lesion are consistent with each other,
the display mode for the first image region is a mode in which the first image region is displayed on the display apparatus and which is determined in accordance with the positional relationship, and
the display mode for the second image region is a mode in which the second image region is displayed on the display apparatus.

5. An image processing apparatus comprising:
a processor, wherein
the processor is configured to:
detect a first image region and a second image region from a medical image obtained by imaging an observation target region including a site of a human body and a lesion, the first image region indicating the site, the second image region indicating the lesion; and
cause a display apparatus to display a result of detection of the first image region and the second image region in a display mode in accordance with a positional relationship between the first image region and the second image region,
the positional relationship is defined by an overlapping degree between the first image region and the second image region.

6. An image processing apparatus comprising:
a processor, wherein
the processor is configured to:
detect a first image region and a second image region from a medical image obtained by imaging an observation target region including a site of a human body and a lesion, the first image region indicating the site, the second image region indicating the lesion; and
cause a display apparatus to display a result of detection of the first image region and the second image region in a display mode in accordance with a positional relationship between the first image region and the second image region,
the positional relationship is defined by an overlapping degree between the first image region and the second image region, and
in a case in which the overlapping degree is greater than or equal to a first degree, the display mode is a mode in which the second image region is displayed so as to be identifiable in the medical image.

7. An image processing apparatus comprising:
a processor, wherein
the processor is configured to:
detect a first image region and a second image region from a medical image obtained by imaging an observation target region including a site of a human body and a lesion, the first image region indicating the site, the second image region indicating the lesion; and
cause a display apparatus to display a result of detection of the first image region and the second image region in a display mode in accordance with a positional relationship between the first image region and the second image region,
the positional relationship is defined by an overlapping degree between the first image region and the second image region, and
in a case in which the overlapping degree is greater than or equal to a first degree, the display mode is a mode in which the second image region is displayed so as to be identifiable in the medical image and the first image region is displayed so as to be comparable with the second image region.

8. An image processing apparatus comprising:
a processor, wherein
the processor is configured to:
detect a first image region and a second image region from a medical image obtained by imaging an observation target region including a site of a human body and a lesion, the first image region indicating the site, the second image region indicating the lesion;
cause a display apparatus to display a result of detection of the first image region and the second image region in a display mode in accordance with a positional relationship between the first image region and the second image region; and
acquire a first certainty factor and a second certainty factor, the first certainty factor being a certainty factor for the result of detection of the first image region, the second certainty factor being a certainty factor for the result of detection of the second image region, and
the display mode is determined in accordance with the first certainty factor, the second certainty factor, and the positional relationship.

9. The image processing apparatus according to claim 8, wherein the display mode is determined in accordance with a magnitude relationship between the first certainty factor and the second certainty factor and the positional relationship.

10. An image processing apparatus comprising:
a processor, wherein
the processor is configured to:
detect a first image region and a second image region from a medical image obtained by imaging an observation target region including a site of a human body and a lesion, the first image region indicating the site, the second image region indicating the lesion; and
cause a display apparatus to display a result of detection of the first image region and the second image region in a display mode in accordance with a positional relationship between the first image region and the second image region,
the display mode is determined in accordance with a plurality of the positional relationships, and
the plurality of the positional relationships are positional relationships between a plurality of the first image regions for a plurality of types of the sites and the second image region.

11. The image processing apparatus according to claim 10, wherein the display mode for each of the plurality of the first image regions differs depending on a corresponding one of the plurality of the positional relationships.

12. The image processing apparatus according to claim 10, wherein the display mode for each of the plurality of the first image regions differs depending on a first image region positional relationship between the plurality of the first image regions.

13. An image processing apparatus comprising:
a processor, wherein
the processor is configured to:
detect a first image region and a second image region from a medical image obtained by imaging an observation target region including a site of a human body and a lesion, the first image region indicating the site, the second image region indicating the lesion; and
cause a display apparatus to display a result of detection of the first image region and the second image region in a display mode in accordance with a positional relationship between the first image region and the second image region,
the medical image is an image defined by a plurality of frames,
the processor is configured to detect the first image region and the second image region for each of the frames, and
the display mode is determined for each of the frames.

14. The image processing apparatus according to claim 13, wherein the processor is configured to:
based on a correspondence relationship between a plurality of types of the sites and a lesion corresponding to each of the sites, determine whether a combination of the first image region and the second image region is correct for each of the frames; and
based on the display mode corresponding to one of the frames used as a determination target in a case in which it is determined that the combination of the first image region and the second image region is correct, correct the display mode corresponding to one of the frames used as a determination target in a case in which it is determined that the combination of the first image region and the second image region is not correct.

15. A medical diagnostic apparatus comprising:
the image processing apparatus according to claim 1; and
an imaging apparatus configured to capture an image of the observation target region.

16. An ultrasonic endoscope apparatus comprising:
the image processing apparatus according to claim 1; and
an ultrasound apparatus configured to acquire an ultrasound image as the medical image.

17. An image processing method comprising:
detecting a first image region and a second image region from a medical image obtained by imaging an observation target region including a site of a human body and a lesion, the first image region indicating the site, the second image region indicating the lesion; and
causing a display apparatus to display a result of detection of the first image region and the second image region in a display mode in accordance with a positional relationship between the first image region and the second image region,
wherein the display mode is determined in accordance with the positional relationship and consistency between the site and the lesion.

18. A non-transitory computer-readable storage medium storing a program executable by a computer to execute a process comprising:
detecting a first image region and a second image region from a medical image obtained by imaging an observation target region including a site of a human body and a lesion, the first image region indicating the site, the second image region indicating the lesion; and
causing a display apparatus to display a result of detection of the first image region and the second image region in a display mode in accordance with a positional relationship between the first image region and the second image region,
the display mode is determined in accordance with the positional relationship and consistency between the site and the lesion.

19. An image processing apparatus comprising:
a processor, wherein
the processor is configured to:
detect a first image region and a second image region from a medical image obtained by imaging an observation target region including a site of a human body and a lesion, the first image region indicating the site, the second image region indicating the lesion; and
determine certainty of the second image region in accordance with a positional relationship between the first image region and the second image region,
the positional relationship is defined by an overlapping degree between the first image region and the second image region.

20. The image processing apparatus according to claim 19, wherein the processor is configured to determine the certainty in accordance with the positional relationship and a relationship between a first certainty factor and a second certainty factor, the first certainty factor being a certainty factor for a result of detection of the first image region, the second certainty factor being a certainty factor for a result of detection of the second image region.

21. An image processing apparatus comprising:
a processor, wherein
the processor is configured to:
detect a first image region and a second image region from a medical image obtained by imaging an observation target region including a site of a human body and a lesion, the first image region indicating the site, the second image region indicating the lesion; and
determine the certainty in accordance with a positional relationship between the first image region and the second image region and a relationship between a first certainty factor and a second certainty factor, the first certainty factor being a certainty factor for a result of detection of the first image region, the second certainty factor being a certainty factor for a result of detection of the second image region.

22. An image processing apparatus comprising:
the processor is configured to:
detect a first image region and a second image region from a medical image obtained by imaging an observation target region including a site of a human body and a lesion, the first image region indicating the site, the second image region indicating the lesion;
determine the certainty in accordance with a positional relationship between the first image region and the second image region and a relationship between a first certainty factor and a second certainty factor, the first certainty factor being a certainty factor for a result of detection of the first image region, the second certainty factor being a certainty factor for a result of detection of the second image region; and
determine that the second image region is certain in a case in which the positional relationship is a preset positional relationship, the first image region and the second image region are not consistent with each other, and the relationship between the first certainty factor and the second certainty factor is a preset certainty factor relationship.

23. An image processing apparatus, comprising:
the processor is configured to:
detect a first image region and a second image region from a medical image obtained by imaging an observation target region including a site of a human body and a lesion, the first image region indicating the site, the second image region indicating the lesion;
determine the certainty in accordance with a positional relationship between the first image region and the second image region; and
determine that the second image region is certain in a case in which the positional relationship is a preset positional relationship and the first image region and the second image region are consistent with each other.

24. The image processing apparatus according to claim 19, wherein the processor is configured to determine certainty of the first image region.

25. The image processing apparatus according to claim 19, wherein the processor is configured to:
cause a display apparatus to display the medical image; and
cause the display apparatus to display information indicating that the lesion is detected if it is determined that the second image region is certain.

26. The image processing apparatus according to claim 25, wherein a position at which the information indicating that the lesion is detected is displayed is a region corresponding to the second image region in a display region in which the medical image is displayed.
